SEQUENCE LISTING (9 sequences)

SEQ ID NO: 1 - protein, 392 aa, Agaricus bisporus
  source 1..392: note = polyphenol oxidase (PPO) enzyme (Agaricus bisporus)
MSDKKSLMPLVGIPGEIKNRLNILDFVKNDKFFTLYVRALQVLQARDQSDYSSFFQLGGIHGLPYTEWAKAQPQLHLYKANYCTHGTVLFPTWHRAYESTWEQTLWEAAGTVAQRFTTSDQAEWIQAAKDLRQPFWDWGYWPNDPDFIGLPDQVIRDKQVEITDYNGTKIEVENPILHYKFHPIEPTFEGDFAQWQTTMRYPDVQKQENIEGMIAGIKAAAPGFREWTFNMLTKNYTWELFSNHGAVVGAHANSLEMVHNTVHFLIGRDPTLDPLVPGHMGSVPHAAFDPIFWMHHCNVDRLLALWQTMNYDVYVSEGMNREATMGLIPGQVLTEDSPLEPFYTKNQDPWQSDDLEDWETLGFSYPDFDPVKGKSKEEKSVYINDWVHKHYG

SEQ ID NO: 2 - protein, 576 aa, Agaricus bisporus
  source 1..576: note = (polyphenol oxidase (PPO) enzyme (Tyrosinase -Agaricus bisporus)
MSDKKSLMPLVGIPGEIKNRLNILDFVKNDKFFTLYVRALQVLQARDQSDYSSFFQLGGIHGLPYTEWAKAQPQLHLYKANYCTHGTVLFPTWHRAYESTWEQTLWEAAGTVAQRFTTSDQAEWIQAAKDLRQPFWDWGYWPNDPDFIGLPDQVIRDKQVEITDYNGTKIEVENPILHYKFHPIEPTFEGDFAQWQTTMRYPDVQKQENIEGMIAGIKAAAPGFREWTFNMLTKNYTWELFSNHGAVVGAHANSLEMVHNTVHFLIGRDPTLDPLVPGHMGSVPHAAFDPIFWMHHCNVDRLLALWQTMNYDVYVSEGMNREATMGLIPGQVLTEDSPLEPFYTKNQDPWQSDDLEDWETLGFSYPDFDPVKGKSKEEKSVYINDWVHKHYGFVTTQTENPALRLLSSFQRAKSDHETQYALYDWVIHATFRYYELNNSFSIIFYFDEGEGCTLESIIGTVDAFRGTTSENCANCARSQDLIAEGFVHLNYYIGCDIGQHADHEDDAVPLYEPTRVKEYLKKRKIGCKVVSAEGELTSLVVEIKGAPYYLPVGEARPKLDHEKPIVILDDIIHRVN

SEQ ID NO: 3 - protein, 576 aa, Agaricus bisporus
  source 1..576: note = (polyphenol oxidase (PPO) enzyme (mutated Tyrosinase N260G -Agaricus bisporus)
MSDKKSLMPLVGIPGEIKNRLNILDFVKNDKFFTLYVRALQVLQARDQSDYSSFFQLGGIHGLPYTEWAKAQPQLHLYKANYCTHGTVLFPTWHRAYESTWEQTLWEAAGTVAQRFTTSDQAEWIQAAKDLRQPFWDWGYWPNDPDFIGLPDQVIRDKQVEITDYNGTKIEVENPILHYKFHPIEPTFEGDFAQWQTTMRYPDVQKQENIEGMIAGIKAAAPGFREWTFNMLTKNYTWELFSNHGAVVGAHANSLEMVHGTVHFLIGRDPTLDPLVPGHMGSVPHAAFDPIFWMHHCNVDRLLALWQTMNYDVYVSEGMNREATMGLIPGQVLTEDSPLEPFYTKNQDPWQSDDLEDWETLGFSYPDFDPVKGKSKEEKSVYINDWVHKHYGFVTTQTENPALRLLSSFQRAKSDHETQYALYDWVIHATFRYYELNNSFSIIFYFDEGEGCTLESIIGTVDAFRGTTSENCANCARSQDLIAEGFVHLNYYIGCDIGQHADHEDDAVPLYEPTRVKEYLKKRKIGCKVVSAEGELTSLVVEIKGAPYYLPVGEARPKLDHEKPIVILDDIIHRVN

SEQ ID NO: 4 - protein, 577 aa, Agaricus bisporus
  source 1..577: note = (polyphenol oxidase (PPO) enzyme (mutated Tyrosinase P159Ins -Agaricus bisporus)
MSDKKSLMPLVGIPGEIKNRLNILDFVKNDKFFTLYVRALQVLQARDQSDYSSFFQLGGIHGLPYTEWAKAQPQLHLYKANYCTHGTVLFPTWHRAYESTWEQTLWEAAGTVAQRFTTSDQAEWIQAAKDLRQPFWDWGYWPNDPDFIGLPDQVIRDKQVEITDYNGTKIEVENPILHYKFHPIEPTFEGDFAQWQTTMRYPDVQKQENIEGMIAGIKAAAPGFREWTFNMLTKNYTWELFSNHGAVVGAHANSLEMVPHNTVHFLIGRDPTLDPLVPGHMGSVPHAAFDPIFWMHHCNVDRLLALWQTMNYDVYVSEGMNREATMGLIPGQVLTEDSPLEPFYTKNQDPWQSDDLEDWETLGFSYPDFDPVKGKSKEEKSVYINDWVHKHYGFVTTQTENPALRLLSSFQRAKSDHETQYALYDWVIHATFRYYELNNSFSIIFYFDEGEGCTLESIIGTVDAFRGTTSENCANCARSQDLIAEGFVHLNYYIGCDIGQHADHEDDAVPLYEPTRVKEYLKKRKIGCKVVSAEGELTSLVVEIKGAPYYLPVGEARPKLDHEKPIVILDDIIHRVN

SEQ ID NO: 5 - protein, 576 aa, Agaricus bisporus
  source 1..576: note = (polyphenol oxidase (PPO) enzyme (mutated Tyrosinase F264R -Agaricus bisporus)
MSDKKSLMPLVGIPGEIKNRLNILDFVKNDKFFTLYVRALQVLQARDQSDYSSFFQLGGIHGLPYTEWAKAQPQLHLYKANYCTHGTVLFPTWHRAYESTWEQTLWEAAGTVAQRFTTSDQAEWIQAAKDLRQPFWDWGYWPNDPDFIGLPDQVIRDKQVEITDYNGTKIEVENPILHYKFHPIEPTFEGDFAQWQTTMRYPDVQKQENIEGMIAGIKAAAPGFREWTFNMLTKNYTWELFSNHGAVVGAHANSLEMVHNTVHRLIGRDPTLDPLVPGHMGSVPHAAFDPIFWMHHCNVDRLLALWQTMNYDVYVSEGMNREATMGLIPGQVLTEDSPLEPFYTKNQDPWQSDDLEDWETLGFSYPDFDPVKGKSKEEKSVYINDWVHKHYGFVTTQTENPALRLLSSFQRAKSDHETQYALYDWVIHATFRYYELNNSFSIIFYFDEGEGCTLESIIGTVDAFRGTTSENCANCARSQDLIAEGFVHLNYYIGCDIGQHADHEDDAVPLYEPTRVKEYLKKRKIGCKVVSAEGELTSLVVEIKGAPYYLPVGEARPKLDHEKPIVILDDIIHRVN

SEQ ID NO: 6 - protein, 270 aa, Portulaca grandiflora
  source 1..270: note = (DOPA 4,5-dioxygenase DODA; Portulaca grandiflora (rose moss)
MGVGKEVSFKESFFLSHGNPAMLADESFIARNFLLGWKKNVFPVKPKSILVVSAHWETDVPCVSAGQYPNVIYDFTEVPASMFQMKYPAPGCPKLAKRVQELLIAGGFKSAKLDEERGFDHSSWVPLSMMCPEADIPVCQLSVQPGLDATHHFNVGRALAPLKGEGVLFIGSGGAVHPSDDTPHWFDGVAPWAAEFDQWEDALLEGRYEDVNNYQTKAPEGWKLAHPIPEHFLPLHVAMGAGGEKSKAELIYRTWDHGTLGYASYKFTSI

SEQ ID NO: 7 - protein, 500 aa, Bombyx mori
  source 1..500: note = (3,4-dihydroxyphenyl-acetaldehyde synthase DHPAA; Bombyx mori (silk moth)
MDANQFREFGRAVIDMLASYAENIRDYDVLPSVEPGYLLRALPESAPEQPEDWKDIMKDFNQSIMPGVTHWQSPQFHAFYPSGSSFASIIGNMLSDGLAVVGFSWMASPACTELEVVTMNWLGKLLDLPEEFLNCSSGPGGGVIQGSASEATLVGLLVAKDKTVRRFMNNNPDLDENEIKAKLVAYTSDQCNSSVEKAGLLGSMKMKLLKADADGCLRGETLKRAIEEDKSQGLIPCYVVANLGTTGTCAFDPLHELGPICSEEDIWLHVDAAYAGAAFLCPEYRHLMKGIEHSQSFVTNAHKWLPVNFDCSAMWVKNGYDITRAFDVQRIYLDDVKTTIKIPDYRHWQMPLGRRFRALKLWTVMRIYGAEGLKTHIRQQIELAQYFAKLVRADERFVIGPEPTMALVCFRLKDGDTITRQLLENITQKKKVFMVAGTHRDRYVIRFVICSRLTKKEDVDYSWSQIKKETDLIYSDKIHNKAQIPALEQFTSRELCEKSK

SEQ ID NO: 8 - protein, 510 aa, Drosophila melanogaster
  source 1..510: note = (3,4-dihydroxyphenyl-acetaldehyde synthase DHPAA; Drosophila melanogaster (fruit)) fly)
MDAKEFREFGKAAIDYIADYLENIRDDDVLPNVEPGYLLDLLPTEMPEEPEAWKDVLGDISRVIKPGLTHWQSPHMHAYYPTSTSYPSIVGEMLASGFGVIGFSWICSPACTELEVVVMDWLAKFLKLPAHFQHASDGPGGGVIQGSASEAVLVAVLAAREQAVANYRESHPELSESEVRGRLVAYSSDQSNSCIEKAGVLAAMPIRLLPAGEDFVLRGDTLRGAIEEDVAAGRIPVICVATLGTTGTCAYDDIESLSAVCEEFKVWLHVDAAYAGGAFALEECSDLRKGLDRVDSLNFNLHKFMLVNFDCSAMWLRDANKVVDSFNVDRIYLKHKHEGQSQIPDFRHWQIPLGRRFRALKVWITFRTLGAEGLRNHVRKHIELAKQFEQLVLKDSRFELVAPRALGLVCFRPKGDNEITTQLLQRLMDRKKIYMVKAEHAGRQFLRFVVCGMDTKASDIDFAWQEIESQLTDLQAEQSLVARKSGNVGDLAQHFQIHLSTENATHEKSQ

SEQ ID NO: 9 - protein, 521 aa, Aedes aegypti
  source 1..521: note = (3,4-dihydroxyphenyl-acetaldehyde synthase DHPAA; Aedes aegypti (mosquito))
MANMDIDEFKEFGKAAIDFVADYLVNIRDRDVLPSVEPGYLHDLLPNEIPEKGDDWKTIMEEFKRFIVPGLTHWQSPHFHAFYPSQTSYSSIVGETLAAGLGVVGFSWICSPVCTELEVIMMNWIGQLLNLPRCFLNCDEGNGGGVIQGSASESIFIAVLVAREQAVRRLKNEHPELTEAEIRGRLVAYTSDQSNSAVEKSGILGAIKMRLLPADDDCVLRGRTLKKAVEEDKANGLFPVIMVATLGTTGTCAYDNLEEIGPYCNDNKLWLHVDAAYAGASFCLPEYAWIKKGLEMADSLNFNLHKWLFVNFDCCAMWFKDAAMITEAFSVDRIYLQHKFQGMSKAPDYRHWQIQLGRRFRSLKVWITLKTMGAEKIRELIRFHISLAQKFEQYVRADPRFEVTSSTLALVCFRLKGEDTYSKQLLDNIVKRKKIYMIPATYQGKFILRFMIAGIDPQAEDIDYAWNEVKSQTDLLLGVDDNGNNVCSKKLIKEEIFEKDNPVGKITESLGGLVLANEKAQ

(19) United States
(12) Patent Application Publication
(10) Pub. No.: US 2025/0176872 A1
(43) Pub. Date: Jun. 5, 2025

(54) SYSTEMS AND METHODS FOR SENSING LEVODOPA

(71) Applicant: DexCom, Inc., San Diego, CA (US)

(72) Inventors: Hifza Najib, San Diego, CA (US); Joshua Ray Windmiller, San Diego, CA (US); Rebekah Pattison Dyer, San Diego, CA (US); Eva Vargas Orgaz, San Diego, CA (US); Nai-Jen Chang, San Diego, CA (US); Jie Zhu, San Diego, CA (US); Matthew Scott Brown, San Diego, CA (US); Jiong Zou, San Diego, CA (US)

(73) Assignee: DexCom, Inc., San Diego, CA (US)

(21) Appl. No.: 18/952,481

(22) Filed: Nov. 19, 2024

Related U.S. Application Data
(60) Provisional application No. 63/605,096, filed on Dec. 1, 2023.
(60) Provisional application No. 63/722,055, filed on Nov. 18, 2024.

(51) Int. Cl. A61B 5/145; A61B 5/1473

(52) U.S. Cl. CPC A61B 5/14546; A61B 5/14735

(57) ABSTRACT

A Levodopa-selective sensor is provided. A continuous levodopa monitor is also provided. The levodopa sensor includes a sensor area having a working electrode with a levodopa-selective chemistry configured for at least partial implantation in a host. The sensor also includes at least one membrane adjacent the levodopa-selective chemistry. The at least one membrane includes an enzyme domain that at least one enzyme selected from a tyrosinase; a mutated tyrosinase with specificity towards 1-3,4-dihydroxyphenylalanine; a dihydroxyphenylalanine 4,5-dioxygenase; a mutated dihydroxyphenylalanine 4,5-dioxygenase with specificity towards 1-3,4-dihydroxyphenylalanine; and combinations thereof; a synthase enzyme with specificity towards 1-3,4-dihydroxyphenylalanine; a mutated synthase enzyme with specificity towards 1-3,4-dihydroxyphenylalanine; a 3,4-dihydroxyphenyl-acetaldehyde synthase; a mutated 3,4-dihydroxyphenyl-acetaldehyde synthase with specificity towards 1-3,4-dihydroxyphenylalanine; a dioxygenase enzyme with specificity towards 1-3,4-dihydroxyphenylalanine; and a mutated dioxygenase enzyme with specificity towards 1-3,4-dihydroxyphenylalanine.

### CROSS-REFERENCE TO RELATED APPLICATIONS

This application claims the benefit of U.S. Provisional Application No. 63/605,096 filed on Dec. 1, 2023, and U.S. Provisional Application No. 63/722,055 filed on Nov. 18, 2024, the entirety of each of which is incorporated herein by reference.

### REFERENCE TO SEQUENCE LISTING

A computer readable XML file entitled “047340.000224.xml”, which was created on Oct. 25, 2024, with a file size of about 15,333 bytes, contains the sequence listing for this application, has been filed with this application, and is hereby incorporated by reference in its entirety.

### TECHNICAL FIELD

This disclosure is directed to devices and methods for monitoring levodopa (L-DOPA) utilizing (mutant) enzymes. This disclosure is also directed to devices and electrochemical methods for continuously monitoring levodopa (L-DOPA) utilizing (mutant) enzymes.

### BACKGROUND

L-DOPA, also known as levodopa and l-3,4-dihydroxyphenylalanine, is currently the most effective drug for treating the symptoms of Parkinson's disease. L-DOPA is a precursor to dopamine and works to increase the amount of dopamine in the brain to manage symptoms. L-DOPA is most effective when administered on a regular basis to maintain the increased amount of dopamine in the brain.

L-DOPA sensors are currently under development to ensure that Parkinson's patients have adequate levels of L-DOPA in their systems. These sensors require an enzyme with high activity solely on the target analyte L-DOPA. Existing commercial enzymes, however, also have a high level of promiscuity, acting upon tyrosine, which is a related and abundant molecule found in humans, thereby precluding the successful construction of a sensor that is specific for L-DOPA rather than structurally or otherwise chemically similar compounds that may co-circulate in the physiologic milieu.

### SUMMARY

In examples, a continuous monitoring sensor is provided, the sensor comprising: an analyte sensing portion comprising a first working electrode, the first working electrode having an electrochemically active surface area, the analyte sensing portion configured to be at least partially implanted; and at least one membrane adjacent the electrochemically active surface area, the at least one membrane comprising an enzyme domain, wherein the enzyme domain comprises at least one L-DOPA responsive enzyme configured to generate at least a first signal corresponding to an L-DOPA concentration.

In aspects, the analyte sensing portion is present on a separate wire substrate or separate planar substrate. In aspects, alone or in combination with any previous aspect, the analyte sensing portion comprises a second working electrode electrically isolated from the first working electrode. In aspects, alone or in combination with any previous aspect, the analyte sensing portion comprises a second working electrode configured to generate a signal associated with a second analyte, the second analyte being chemically different from the first analyte. In aspects, alone or in combination with any previous aspect, the analyte sensing portion comprises a second working electrode electrically isolated from the first working electrode without the at least one L-DOPA responsive enzyme.

In aspects, alone or in combination with any previous aspect, the analyte sensing portion comprises a second working electrode electrically isolated from the first working electrode configured for detecting oxygen. In aspects, alone or in combination with any previous aspect, the analyte sensing portion comprises a second working electrode configured to generate a signal associated with a measurement of in situ electroactive species.

In aspects, alone or in combination with any previous aspect, the at least one membrane comprises one or more layers. In aspects, alone or in combination with any previous aspect, the one or more layers is configured for buffering of the enzyme domain. In aspects, alone or in combination with any previous aspect, one or more layers configured for protonation of L-dopaquinone. In aspects, alone or in combination with any previous aspect, the one or more layers configured for attenuation of signal-interfering electroactive species to the electrochemically active surface area. In aspects, alone or in combination with any previous aspect, the one or more layers, independently, are configured for protonation of L-dopaquinone and oxygen diffusion to the electrochemically active surface area. In aspects, alone or in combination with any previous aspect, the one or more layers, independently, are configured for protonation of L-dopaquinone, oxygen diffusion, and attenuation of signal-interfering electroactive species to the electrochemically active surface area.

In aspects, alone or in combination with any previous aspect, the at least one membrane or the one or more layers independently comprise a polymer chain having both hydrophilic and hydrophobic regions. In aspects, alone or in combination with any previous aspect, the at least one membrane or the one or more layers independently comprise a polymer chain having polyurethane and/or polyurea segments.

In aspects, alone or in combination with any previous aspect, the polyurethane and/or polyurea segments comprise hard segments and soft segments. In aspects, alone or in combination with any previous aspect, the soft segments comprise poly(tetramethylene oxide) repeating units. In aspects, alone or in combination with any previous aspect, the soft segments comprise polydialkylsiloxane repeating units. In aspects, alone or in combination with any previous aspect, the soft segments comprise both poly(tetramethylene oxide) repeating units and polydialkylsiloxane repeating units.

In aspects, alone or in combination with any previous aspect, the at least one membrane or the one or more layers independently comprise a polyurethane and/or polyurea polymer blended with polyvinylpyrrolidone.

In aspects, alone or in combination with any previous aspect, the at least one membrane or the one or more layers independently comprise a polymer or polymerized monomer comprising a zwitterionic functional group. In aspects, alone or in combination with any previous aspect, the zwitterionic functional group is configured for protonation of L-dopaquinone present about the analyte sensing portion.

In aspects, alone or in combination with any previous aspect, the at least one membrane or the one or more layers independently comprise a coating comprising a polymer with a styrene group. In aspects, alone or in combination with any previous aspect, the at least one membrane or the one or more layers independently comprise a coating comprising a polymer with a heterocyclic group. In aspects, alone or in combination with any previous aspect, the at least one membrane or the one or more layers independently comprise a coating comprising a polymer chain having poly(1-vinyl imidazole), poly(4-vinyl pyridine), poly(2-vinyl pyridine), acrylonitrile, acrylamide, and/or copolymers or quaternized forms thereof.

In aspects, alone or in combination with any previous aspect, the at least one L-DOPA responsive enzyme is a tyrosinase enzyme with specificity towards l-3,4-dihydroxyphenylalanine (L-DOPA). In aspects, alone or in combination with any previous aspect, the at least one L-DOPA responsive enzyme is a mutated tyrosinase enzyme with specificity towards l-3,4-dihydroxyphenylalanine (L-DOPA).

In aspects, alone or in combination with any previous aspect, the mutant enzyme has at least 90% identity to: amino acids 1-392 of SEQ ID NO: 1; amino acids 1-576 of SEQ ID NO: 2; amino acids 1-576 of SEQ ID NO: 3; amino acids 1-577 of SEQ ID NO: 4, or amino acids 1-576 of SEQ ID NO: 5.

In aspects, alone or in combination with any previous aspect, the substitution in the polypeptide chain occurs at a position that corresponds to an amino acid residue position H61 through H296 of SEQ ID NOs: 1-4. In aspects, alone or in combination with any previous aspect, the substitution in the polypeptide chain excludes a position that corresponds to an amino acid residue position H61 through H296 of SEQ ID NOs: 1-4.

In aspects, alone or in combination with any previous aspect, the at least one L-DOPA responsive enzyme is a dioxygenase enzyme with specificity towards l-3,4-dihydroxyphenylalanine (L-DOPA). In aspects, alone or in combination with any previous aspect, the at least one L-DOPA responsive enzyme is a mutated dioxygenase enzyme with specificity towards l-3,4-dihydroxyphenylalanine (L-DOPA).

In aspects, alone or in combination with any previous aspect, the at least one L-DOPA responsive enzyme is a DOPA 4,5-dioxygenase with specificity towards l-3,4-dihydroxyphenylalanine (L-DOPA). In aspects, alone or in combination with any previous aspect, the at least one L-DOPA responsive enzyme is a mutated DOPA 4,5-dioxygenase with specificity towards l-3,4-dihydroxyphenylalanine (L-DOPA).

In aspects, alone or in combination with any previous aspect, the mutated DOPA 4,5-dioxygenase comprises a substitution in the polypeptide chain at a position that corresponds to an amino acid residue position H17 through H232 of SEQ ID NO: 6. In aspects, alone or in combination with any previous aspect, the mutated DOPA 4,5-dioxygenase comprises a substitution in the polypeptide chain at a position that corresponds to an amino acid residue position H17 through H232 of SEQ ID NO: 6. In aspects, alone or in combination with any previous aspect, the mutated DOPA 4,5-dioxygenase comprises a substitution in the polypeptide chain at a position that corresponds to one or more amino acid residue positions selected from H17, H55, H177, P178, S179, D180, and H232 of SEQ ID NO: 6. In aspects, alone or in combination with any previous aspect, the mutated DOPA 4,5-dioxygenase comprises a substitution in the polypeptide chain at a position that corresponds to one or more amino acid residue positions selected from H17, H55, H177, P178, S179, D180, and H232 of SEQ ID NO: 6.

In aspects, alone or in combination with any previous aspect, the at least one L-DOPA responsive enzyme is a synthase enzyme with specificity towards l-3,4-dihydroxyphenylalanine (L-DOPA). In aspects, alone or in combination with any previous aspect, the at least one L-DOPA responsive enzyme is a mutated synthase enzyme with specificity towards l-3,4-dihydroxyphenylalanine (L-DOPA).

In aspects, alone or in combination with any previous aspect, the at least one L-DOPA responsive enzyme is a 3,4-dihydroxyphenyl-acetaldehyde synthase. In aspects, alone or in combination with any previous aspect, the at least one L-DOPA responsive enzyme is a mutated 3,4-dihydroxyphenyl-acetaldehyde synthase with specificity towards l-3,4-dihydroxyphenylalanine (L-DOPA).

In aspects, alone or in combination with any previous aspect, the analyte sensing portion comprises a mutant 3,4-dihydroxyphenyl-acetaldehyde synthase enzyme wherein the substitution in the polypeptide chain occurs at a position that corresponds to an amino acid residue position Y80 through K303 (PLP) of SEQ ID NO: 7 or SEQ ID NO: 8. In aspects, alone or in combination with any previous aspect, the substitution in the polypeptide chain excludes a position that corresponds to an amino acid residue position Y80 through K303 (PLP) of SEQ ID NO: 7 or SEQ ID NO: 8. In aspects, alone or in combination with any previous aspect, the substitution in the polypeptide chain occurs at a position that corresponds to one or more amino acid residue positions selected from Y80, S147, N192, and K303 (PLP) of SEQ ID NO: 7 or SEQ ID NO: 8. In aspects, alone or in combination with any previous aspect, the substitution in the polypeptide chain excludes a position that corresponds to one or more amino acid residue positions selected from Y80, S147, N192, and K303 (PLP) of SEQ ID NO: 7 or SEQ ID NO: 8.

In aspects, alone or in combination with any previous aspect, the substitution in the polypeptide chain occurs at a position that corresponds to an amino acid residue position Y83 through K306 (PLP) of SEQ ID NO: 9. In aspects, alone or in combination with any previous aspect, the substitution in the polypeptide chain excludes a position that corresponds to an amino acid residue position Y83 through K306 (PLP) of SEQ ID NO: 9. In aspects, alone or in combination with any previous aspect, the substitution in the polypeptide chain occurs at a position that corresponds to one or more amino acid residue positions selected from Y83, S150, N195, and K306 (PLP) of SEQ ID NO: 9. In aspects, alone or in combination with any previous aspect, the substitution in the polypeptide chain excludes a position that corresponds to one or more amino acid residue positions selected from Y83, S150, N195, and K306 (PLP) of SEQ ID NO: 9.

In examples, a method of continuously monitoring levodopa is provided, the method comprising: exposing an analyte sensing portion of an analyte sensor comprising at least one enzyme to a biological fluid of a subject, the at least one enzyme selected from: a tyrosinase; a mutated tyrosinase with specificity towards l-3,4-dihydroxyphenylalanine; a dihydroxyphenylalanine 4,5-dioxygenase; a mutated dihydroxyphenylalanine 4,5-dioxygenase with specificity towards l-3,4-dihydroxyphenylalanine; and combinations thereof; a synthase enzyme with specificity towards l-3,4-dihydroxyphenylalanine; a mutated synthase enzyme with specificity towards l-3,4-dihydroxyphenylalanine; a 3,4-dihydroxyphenyl-acetaldehyde synthase; a mutated 3,4-dihydroxyphenyl-acetaldehyde synthase with specificity towards l-3,4-dihydroxyphenylalanine; a dioxygenase enzyme with specificity towards l-3,4-dihydroxyphenylalanine; a mutated dioxygenase enzyme with specificity towards l-3,4-dihydroxyphenylalanine; and detecting an electrochemical signal corresponding to a levodopa concentration in the biological fluid.

In aspects, the analyte sensing portion is present on a separate wire substrate or separate planar substrate. In aspects, alone or in combination with any previous aspect, the analyte sensing portion comprises a second working electrode electrically isolated from the first working electrode. In aspects, alone or in combination with any previous aspect, the analyte sensing portion comprises a second working electrode configured to generate a signal associated with a second analyte, the second analyte being chemically different from the first analyte. In aspects, alone or in combination with any previous aspect, the analyte sensing portion comprises a second working electrode electrically isolated from the first working electrode without the at least one L-DOPA responsive enzyme.

In aspects, alone or in combination with any previous aspect, the analyte sensing portion comprises a second working electrode electrically isolated from the first working electrode configured for detecting oxygen. In aspects, alone or in combination with any previous aspect, the analyte sensing portion comprises a second working electrode configured to generate a signal associated with a measurement of in situ electroactive species.

In aspects, alone or in combination with any previous aspect, the analyte sensing portion further comprises at least one membrane or one or more layers. In aspects, alone or in combination with any previous aspect, the at least one membrane or the one or more layers independently comprise a polymer chain having both hydrophilic and hydrophobic regions.

In aspects, alone or in combination with any previous aspect, the at least one membrane or the one or more layers independently comprise a polymer chain having polyurethane and/or polyurea segments. In aspects, alone or in combination with any previous aspect, the polyurethane and/or polyurea segments comprise hard segments and soft segments. In aspects, alone or in combination with any previous aspect, the soft segments comprise poly(tetramethylene oxide) repeating units. In aspects, alone or in combination with any previous aspect, the soft segments comprise polydialkylsiloxane repeating units. In aspects, alone or in combination with any previous aspect, the soft segments comprise both poly(tetramethylene oxide) repeating units and polydialkylsiloxane repeating units.

In aspects, alone or in combination with any previous aspect, the at least one membrane or the one or more layers independently comprise a polyurethane and/or polyurea polymer blended with polyvinylpyrrolidone.

In aspects, alone or in combination with any previous aspect, the at least one membrane or the one or more layers independently comprise a polymer or polymerized monomer comprising a zwitterionic functional group. In aspects, alone or in combination with any previous aspect, the zwitterionic functional group protonates L-dopaquinone present about the analyte sensing portion.

In aspects, alone or in combination with any previous aspect, the at least one membrane or the one or more layers independently comprise a coating comprising a polymer with a styrene group. In aspects, alone or in combination with any previous aspect, the at least one membrane or the one or more layers independently comprise a coating comprising a polymer with a heterocyclic group.

In aspects, alone or in combination with any previous aspect, the at least one membrane or the one or more layers independently comprise a coating comprising a polymer chain having poly(1-vinyl imidazole), poly(4-vinyl pyridine), poly(2-vinyl pyridine), acrylonitrile, acrylamide, and/or copolymers or quaternized forms thereof.

In aspects, alone or in combination with any previous aspect, the mutant tryrosinase has at least 90% identity to: amino acids 1-392 of SEQ ID NO: 1; amino acids 1-576 of SEQ ID NO: 2; amino acids 1-576 of SEQ ID NO: 3; amino acids 1-577 of SEQ ID NO: 4, or amino acids 1-576 of SEQ ID NO: 5.

In aspects, alone or in combination with any previous aspect, the substitution in the polypeptide chain occurs at a position that corresponds to an amino acid residue position H61 through H296 of SEQ ID NOs: 1-4. In aspects, alone or in combination with any previous aspect, the substitution in the polypeptide chain excludes a position that corresponds to an amino acid residue position H61 through H296 of SEQ ID NOs: 1-4.

In aspects, alone or in combination with any previous aspect, the at least one L-DOPA responsive enzyme is a dioxygenase enzyme with specificity towards l-3,4-dihydroxyphenylalanine (L-DOPA). In aspects, alone or in combination with any previous aspect, the at least one L-DOPA responsive enzyme is a mutated dioxygenase enzyme with specificity towards l-3,4-dihydroxyphenylalanine (L-DOPA).

In aspects, alone or in combination with any previous aspect, the at least one L-DOPA responsive enzyme is a DOPA 4,5-dioxygenase. In aspects, alone or in combination with any previous aspect, the at least one L-DOPA responsive enzyme is a mutated DOPA 4,5-dioxygenase with specificity towards l-3,4-dihydroxyphenylalanine (L-DOPA).

In aspects, alone or in combination with any previous aspect, the mutated DOPA 4,5-dioxygenase comprises a substitution in the polypeptide chain at a position that corresponds to an amino acid residue position H17 through H232 of SEQ ID NO: 6. In aspects, alone or in combination with any previous aspect, the mutated DOPA 4,5-dioxygenase comprises a substitution in the polypeptide chain at a position that corresponds to an amino acid residue position H17 through H232 of SEQ ID NO: 6.

In aspects, alone or in combination with any previous aspect, the mutated DOPA 4,5-dioxygenase comprises a substitution in the polypeptide chain at a position that corresponds to one or more amino acid residue positions selected from H17, H55, H177, P178, S179, D180, and H232 of SEQ ID NO: 6. In aspects, alone or in combination with any previous aspect, the mutated DOPA 4,5-dioxygenase comprises a substitution in the polypeptide chain at a position that corresponds to one or more amino acid residue positions selected from H17, H55, H177, P178, S179, D180, and H232 of SEQ ID NO: 6.

In aspects, alone or in combination with any previous aspect, the synthase enzyme or the mutated synthase enzyme with specificity towards l-3,4-dihydroxyphenylalanine comprises a 3,4-dihydroxyphenyl-acetaldehyde synthase enzyme. In aspects, alone or in combination with any previous aspect, the synthase enzyme or the mutated synthase enzyme with specificity towards l-3,4-dihydroxyphenylalanine comprises a mutant 3,4-dihydroxyphenyl-acetaldehyde synthase enzyme.

In aspects, alone or in combination with any previous aspect, the mutant 3,4-dihydroxyphenyl-acetaldehyde synthase enzyme comprises a substitution in the polypeptide chain at a position that corresponds to an amino acid residue position Y80 through K303 (PLP) of SEQ ID NO: 7 or SEQ ID NO: 8. In aspects, alone or in combination with any previous aspect, the substitution in the polypeptide chain excludes a position that corresponds to an amino acid residue position Y80 through K303 (PLP) of SEQ ID NO: 7 or SEQ ID NO: 8. In aspects, alone or in combination with any previous aspect, the substitution in the polypeptide chain occurs at a position that corresponds to one or more amino acid residue positions selected from Y80, S147, N192, and K303 (PLP) of SEQ ID NO: 7 or SEQ ID NO: 8. In aspects, alone or in combination with any previous aspect, the substitution in the polypeptide chain excludes a position that corresponds to one or more amino acid residue positions selected from Y80, S147, N192, and K303 (PLP) of SEQ ID NO: 7 or SEQ ID NO: 8. In aspects, alone or in combination with any previous aspect, the substitution in the polypeptide chain occurs at a position that corresponds to an amino acid residue position Y83 through K306 (PLP) of SEQ ID NO: 9. In aspects, alone or in combination with any previous aspect, the substitution in the polypeptide chain excludes a position that corresponds to an amino acid residue position Y83 through K306 (PLP) of SEQ ID NO: 9. In aspects, alone or in combination with any previous aspect, the substitution in the polypeptide chain occurs at a position that corresponds to one or more amino acid residue positions selected from Y83, S150, N195, and K306 (PLP) of SEQ ID NO: 9. In aspects, alone or in combination with any previous aspect, the substitution in the polypeptide chain excludes a position that corresponds to one or more amino acid residue positions selected from Y83, S150, N195, and K306 (PLP) of SEQ ID NO: 9.

In other examples, a method of operating an analyte sensor for detecting levodopa in a fluid is provided, the analyte sensor comprising: at least a first working electrode; an analyte sensing portion disposed on a surface of the first working electrode, the analyte sensing portion configured for introduction to interstitial fluid (ISF), the analyte sensing portion capable of at least facilitating detection of levodopa; the method comprising: applying a potential to the first working electrode at or above an oxidation-reduction potential of the analyte sensing portion to generate a signal corresponding to the levodopa concentration in the ISF; and correlating the signal to the levodopa concentration in the ISF.

### BRIEF DESCRIPTION OF THE DRAWINGS

In order to understand and to see how the present disclosure may be carried out in practice, examples will now be described, by way of non-limiting examples only, with reference to the accompanying drawings, in which:

FIG. 1A illustrates mutant enzyme activity in accordance with the broadest aspect of the present disclosure.

FIG. 1B illustrates mutant enzyme activity in accordance with the broadest aspect of the present disclosure in accordance with the broadest aspect of the present disclosure.

FIG. 1C illustrates mutant enzyme diphenol specificity in accordance with the broadest aspect of the present disclosure.

FIG. 1D illustrates a tyrosinase-based L-DOPA sensor in accordance with the broadest aspect of the present disclosure.

FIG. 2A illustrates a synthase-based L-DOPA sensor in accordance with the broadest aspect of the present disclosure.

FIG. 2B illustrates a cascade synthase-tyrosinase based L-DOPA sensor in accordance with the broadest aspect of the present disclosure.

FIG. 3 illustrates a dioxygenase-based L-DOPA sensor in accordance with the broadest aspect of the present disclosure.

FIG. 4 is a diagram illustrating certain embodiments of an example continuous transcutaneous analyte sensor system communicating with at least one display device in accordance with various technologies described in the present disclosure.

FIG. 5 is a diagram showing one example of a medical device system including the analyte sensor system of FIG. 4.

FIG. 6A is an illustration of an example analyte sensor in accordance with the broadest aspect of the present disclosure.

FIG. 6B is an enlarged view of an example analyte sensor of the analyte sensor system shown in FIG. 6A.

FIG. 6C is a cross-sectional view of the analyte sensor of FIG. 6B.

FIG. 7A is a perspective-view schematic illustrating an in vivo portion of an exemplary continuous analyte sensor.

FIG. 7B is an expanded perspective schematic of section 7B the distal portion of the sensor example illustrated in FIG. 7A.

FIG. 7C is a graphical schematic illustrating oxygen adjustment of an exemplary continuous analyte sensor.

FIG. 8 is an illustration of an example planar analyte sensor with sensing membranes in accordance with the broadest aspect of the present disclosure.

FIGS. 9A-9E illustrate a double-sided, co-planar un-connected analyte sensor, in accordance with an example.

FIGS. 10A-10E illustrate a double-sided, co-planar connected analyte sensor, in accordance with an example.

### DETAILED DESCRIPTION

Mutant enzymes, devices, and methods are herein disclosed and described to provide diphenol specificity and continuous analyte sensing in order to detect a desired analyte in a biological fluid.

In order to facilitate an understanding of the disclosed examples, a number of terms are defined below.

The terms and phrases “analyte measuring device,” “analyte sensing device,” “biosensor,” “sensor,” “sensing region,” “sensing portion,” and “sensing mechanism” as used herein are broad terms and phrases, and are to be given their ordinary and customary meaning to a person of ordinary skill in the art (and are not to be limited to a special or customized meaning), and refer without limitation to the area of an analyte-monitoring device responsible for the detection of, or transduction of a signal associated with, a particular analyte or combination of analytes. For example, those terms may refer without limitation to the region of a monitoring device responsible for the detection of a particular analyte. In examples, sensing region generally comprises a non-conductive body, a working electrode (anode), a reference electrode (optional), and/or a counter electrode (cathode) passing through and secured within the body forming electrochemically reactive surfaces on the body and an electronic connective means at another location on the body, and a multi-domain membrane affixed to the body and covering the electrochemically reactive surface. In examples, such devices are capable of providing specific quantitative, semi-quantitative, qualitative, semi qualitative analytical information using a biological recognition element combined with a transducing (detecting) element.

The term “about” as used herein is a broad term, and is to be given its ordinary and customary meaning to a person of ordinary skill in the art (and is not be limited to a special or customized meaning), and refers without limitation to allowing for a degree of variability in a value or range, for example, within 10%, within 5%, or within 1% of a stated value or of a stated limit of a range, and includes the exact stated value or range. The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The phrase “substantially free of” as used herein can mean having none or having a trivial amount of, such that the amount of material present does not affect the material properties of the composition including the material, such that about 0 wt. % to about 5 wt. % of the composition is the material, or about 0 wt. % to about 1 wt. %, or about 5 wt. % or less, or less than or equal to about 4.5 wt. %, 4, 3.5, 3, 2.5, 2, 1.5, 1, 0.9, 0.8, 0.7, 0.6, 0.5, 0.4, 0.3, 0.2, 0.1, 0.01, or about 0.001 wt. % or less, or about 0 wt. %.

The term “accuracy” as used herein are broad terms, and are to be given their ordinary and customary meaning to a person of ordinary skill in the art (and are not be limited to a special or customized meaning), and refers to a mean absolute relative difference of no more than 20% over a period of time the sensor is applied to (e.g., implanted in) the host or is being used to obtain sensor values for an analyte, wherein one or more reference measurements associated with calculation of the mean absolute relative difference are determined by analysis of blood for the same analyte.

The terms “adhere” and “attach” as used herein are broad terms, and are to be given their ordinary and customary meaning to a person of ordinary skill in the art (and are not be limited to a special or customized meaning), and refer without limitation to hold, bind, or stick, for example, by gluing, bonding, grasping, interpenetrating, or fusing.

The term “analyte” as used herein is a broad term, and is to be given its ordinary and customary meaning to a person of ordinary skill in the art (and is not to be limited to a special or customized meaning), and refers without limitation to a substance or chemical constituent in a biological fluid (e.g., blood, interstitial fluid, cerebral spinal fluid, lymph fluid, urine, sweat, saliva, etc.) that can be analyzed. Analytes can include naturally occurring substances, artificial substances, metabolites, and/or reaction products. In some examples, the analyte measured by the sensing regions, devices, and methods is l-3,4-dihydroxyphenylalanine (levodopa or L-DOPA). However, other analytes are contemplated as well, including but not limited to acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); bilirubin, biotinidase; biopterin; c-reactive protein; carnitine; carnosinase; CD4; ceruloplasmin; chenodeoxycholic acid; chloroquine; cholesterol; cholinesterase; conjugated 1-β hydroxy-cholic acid; cortisol; creatine; creatine kinase; creatine kinase MM isoenzyme; creatinine; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, glucose-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose; glutathione; glutathione perioxidase; glycerol; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; beta-hydroxybutyrate; ketones; lactate; lead; lipoproteins ((a), B/A-1, β); lysozyme; mefloquine; netilmicin; oxygen; phenobarbitone; phenytoin; phytanic/pristanic acid; potassium, sodium, and/or other blood electrolytes; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uric acid; uroporphyrinogen I synthase; vitamin A; white blood cells; and zinc protoporphyrin. Salts, sugar, protein, fat, vitamins, and hormones naturally occurring in blood or interstitial fluids can also constitute analytes in certain examples. The analyte can be naturally present in the biological fluid, or endogenous, for example, a metabolic product, a hormone, an antigen, an antibody, and the like. Alternately, the analyte can be introduced into the body, or exogenous, for example, a contrast agent for imaging, a radioisotope, a chemical agent, a fluorocarbon-based synthetic blood, or a drug or pharmaceutical composition, including but not limited to insulin; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbiturates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-dihydroxyphenylacetic acid (DOPAC), homovanillic acid (HVA), 5-hydroxytryptamine (5HT) (serotonin), 5-hydroxyindoleacetic acid (FHIAA), and histamine.

The term “bioactive agent” as used herein is a broad term, and is to be given its ordinary and customary meaning to a person of ordinary skill in the art (and is not to be limited to a special or customized meaning), and refers without limitation to any substance that has an effect on or elicits a response from living tissue.

The phrases “biointerface membrane” and “biointerface layer” as used interchangeably herein are broad phrases, and are to be given their ordinary and customary meaning to a person of ordinary skill in the art and are not to be limited to a special or customized meaning, and refer without limitation to a permeable membrane, which can include multiple domains or layer that functions as a bioprotective interface between host tissue and an implantable device. The terms “biointerface,” “biocompatible” and “bioprotective” are used interchangeably herein.

The term “continuous” as used herein is a broad term, and is to be given its ordinary and customary meaning to a person of ordinary skill in the art (and is not to be limited to a special or customized meaning), and refers without limitation to an uninterrupted or unbroken portion, domain, coating, or layer.

The phrase “continuous analyte sensing” as used herein is a broad phrase, and is to be given its ordinary and customary meaning to a person of ordinary skill in the art (and is not to be limited to a special or customized meaning), and refers without limitation to the period in which monitoring of concentration of an analyte is continuously, continually, and/or intermittently (but regularly) performed, for example, from about every 5 seconds or less to about 10 minutes or more. In further examples, monitoring of concentration of an analyte is performed from about every 10, 15, 20, 25, 30, 35, 40, 45, 50, 55, or 60 second to about 1.25, 1.50, 1.75, 2.00, 2.25, 2.50, 2.75, 3.00, 3.25, 3.50, 3.75, 4.00, 4.25, 4.50, 4.75, 5.00, 5.25, 5.50, 5.75, 6.00, 6.25, 6.50, 6.75, 7.00, 7.25, 7.50, 7.75, 8.00, 8.25, 8.50, 8.75, 9.00, 9.25, 9.50 or 9.75 minutes.

The term “coupled” as used herein is a broad term, and is to be given its ordinary and customary meaning to a person of ordinary skill in the art (and is not to be limited to a special or customized meaning), and refers without limitation to two or more system elements or components that are configured to be at least one of electrically, mechanically, thermally, operably, chemically or otherwise attached. Similarly, the phrases “operably connected”, “operably linked”, and “operably coupled” as used herein may refer to one or more components linked to another component(s) in a manner that facilitates transmission of at least one signal between the components. In some examples, components are part of the same structure and/or integral with one another (i.e. “directly coupled”). In other examples, components are connected via remote means. For example, one or more electrodes can be used to detect an analyte in a sample and convert that information into a signal; the signal can then be transmitted to an electronic circuit. In this example, the electrode is “operably linked” to the electronic circuit. The phrase “removably coupled” as used herein may refer to two or more system elements or components that are configured to be or have been electrically, mechanically, thermally, operably, chemically, or otherwise attached and detached without damaging any of the coupled elements or components. The phrase “permanently coupled” as used herein may refer to two or more system elements or components that are configured to be or have been electrically, mechanically, thermally, operably, chemically, or otherwise attached but cannot be uncoupled without damaging at least one of the coupled elements or components.

The term “distal” as used herein is a broad term, and is to be given its ordinary and customary meaning to a person of ordinary skill in the art (and is not to be limited to a special or customized meaning), and refers without limitation to a region spaced relatively far from a point of reference, such as an origin or a point of attachment.

The term “domain” as used herein is a broad term, and is to be given its ordinary and customary meaning to a person of ordinary skill in the art (and is not to be limited to a special or customized meaning), and refers without limitation to a region of the membrane system that can be a layer, a uniform or non-uniform gradient (for example, an anisotropic region of a membrane), or a portion of a membrane that is capable of sensing one, two, or more analytes. The domains discussed herein can be formed as a single layer, as two or more layers, as pairs of bi-layers, or as combinations thereof.

The term “drift” as used herein is a broad term, and is to be given its ordinary and customary meaning to a person of ordinary skill in the art (and is not to be limited to a special or customized meaning), and refers without limitation to a progressive increase or decrease in signal over time that is unrelated to changes in host systemic analyte concentrations. While not wishing to be bound by theory, it is believed that drift may be the result of a local decrease in L-DOPA transport to the sensor, for example, due to a formation of a foreign body capsule (FBC). It is also believed that an insufficient amount of interstitial fluid surrounding the sensor may result in reduced oxygen and/or in L-DOPA transport to the sensor. In examples, an increase in local interstitial fluid may slow or reduce drift and thus improve sensor performance. Drift may also be the result of sensor electronics, or algorithmic models used to compensate for noise or other anomalies that can occur with electrical signals in ranges including the microampere range, picoampere range, nanoampere range, and femtoampere range.

The phrases “drug releasing membrane” and “drug releasing layer” as used interchangeably herein are each a broad phrase, and each are to be given its ordinary and customary meaning to a person of ordinary skill in the art (and is not to be limited to a special or customized meaning), and refers without limitation to a permeable or semi-permeable membrane which is permeable to one or more bioactive agents. In examples, the “drug releasing membrane” and “drug releasing layer” can be comprised of two or more domains and is typically of a few microns thickness or more. In examples the drug releasing layer and/or drug releasing membrane are substantially the same as the biointerface layer and/or biointerface membrane. In another example, the drug releasing layer and/or drug releasing membrane are distinct from the biointerface layer and/or biointerface membrane. Further examples of drug releasing layers and membranes may be found in pending U.S. Provisional Application No. Application Number: 63/318,901, titled “DRUG RELEASING MEMBRANE FOR ANALYTE SENSOR,” filed Mar. 11, 2022, incorporated by reference in its entirety herein.

The phrases “electrochemically reactive surface” and “electrochemically active surface” as used herein interchangeably and are broad phrases, and are to be given its ordinary and customary meaning to a person of ordinary skill in the art (and is not to be limited to a special or customized meaning), and refers without limitation to the surface of an electrode where an electrochemical reaction takes place. Electrodes with electrochemically active surfaces include platinum and its binary and tertiary alloys, palladium and its binary and tertiary alloys, gold and its binary and tertiary alloys, silver and its binary and tertiary alloys, iridium and its binary and tertiary alloys, rhodium and its binary and tertiary alloys, nitinol, indium tin oxide, bismuth molybdate (Bi₂MoO₆), tin sulfide metal oxide (SnS₂), boron doped diamond, platinum coated boron doped diamond, conductive graphite and inks therefrom, gold, platinum, pallidum or iridium coated silicon wafers, doped polyaniline, doped poly(3,4-ethylenedioxythio-phene) polystyrene sulfonate (PEDOT:PSS), doped polypyrrole (Ppy), amorphous carbon, carbon nanotubes, graphene metallic nanoparticles, and/or ternary metal oxide composites. In examples, dopaquinone produced by an enzyme-catalyzed reaction of an analyte being detected reacts can create a measurable electronic current. in another example, electron transfer is provided using a mediator or “wired enzyme” during reduction-oxidation (redox) of the transducing element and the analyte.

The terms “implanted” or “implantable” as used herein are broad terms, and are to be given their ordinary and customary meaning to a person of ordinary skill in the art (and are not to be limited to a special or customized meaning), and refer without limitation to objects (e.g., sensors) that are inserted intracutaneously (i.e. in the epidermis or dermis), subcutaneously (i.e. in the layer of fat between the skin and the muscle) or transcutaneously (i.e. penetrating, entering, passing through intact skin, or passing through the top layer of skin (stratum corneum)), which may result in a sensor that has an in vivo portion and an ex vivo portion. The terms “implanted” or “implantable” as used herein encompasses indwelling sensors.

The term “indwell” or “indwelling,” as used herein is a broad term, and is to be given its ordinary and customary meaning to a person of ordinary skill in the art (and are not to be limited to a special or customized meaning), and refers without limitation to reside within a host's body. Some medical devices can indwell within a host's body for various lengths of time, depending upon the purpose of the medical device, such as but not limited to minutes, a few hours, days, weeks, to months, years, or even the host's entire lifetime. In some examples, indwelling medical devices can be removed, for example, without surgical intervention.

The terms “interferents” and “interfering species” as used herein are broad terms, and are to be given their ordinary and customary meaning to a person of ordinary skill in the art (and are not to be limited to a special or customized meaning), and refer without limitation to effects and/or species that interfere with the measurement of an analyte of interest in a sensor to produce a signal that does not accurately represent the analyte measurement. In examples of an electrochemical sensor, interfering species are compounds with an oxidation potential that overlaps with the analyte to be measured or one or more mediators.

The term “in vivo” as used herein is a broad term, and is to be given its ordinary and customary meaning to a person of ordinary skill in the art (and is not to be limited to a special or customized meaning), and without limitation is inclusive of the portion of a device (for example, a sensor) adapted for insertion into and/or existence within a living body of a host.

The term “ex vivo” as used herein is a broad term, and is to be given its ordinary and customary meaning to a person of ordinary skill in the art (and is not to be limited to a special or customized meaning), and without limitation is inclusive of a portion of a device (for example, a sensor) adapted to remain and/or exist outside of a living body of a host.

The term and phrase “mediator” and “redox mediator” as used herein are broad terms and phrases, and are to be given their ordinary and customary meaning to a person of ordinary skill in the art (and is not to be limited to a special or customized meaning), and refers without limitation to any chemical compound or collection of compounds capable of electron transfer, either directly, or indirectly, between an analyte, analyte precursor, analyte surrogate, analyte-reduced or analyte-oxidized enzyme, or cofactor, and an electrode surface held at a potential. In examples the mediator accepts electrons from, or transfer electrons to, one or more enzymes or cofactors, and/or exchanges electrons with the sensor system electrodes. In examples, mediators include electroreducible and electrooxidizable ions or complexes having oxidation-reduction potentials above or below the oxidation-reduction potential of a standard calomel electrode (SCE), for example, about 50, 75, 100, 150, 200, 250, 300, 350, 400, 450, 500, 550, 600, 650, 700, or 750 millivolts above or below the oxidation-reduction potential of the SCE are transition-metal coordinated organic molecules which are capable of reversible oxidation and reduction reactions. In other examples, mediators are organic molecules or metals which are capable of reversible oxidation and reduction reactions.

The term “membrane” as used herein is a broad term, and is to be given its ordinary and customary meaning to a person of ordinary skill in the art (and is not to be limited to a special or customized meaning), and refers without limitation to a structure configured to perform functions including, but not limited to, protection of the exposed electrode surface from the biological environment, diffusion resistance (limitation) of the analyte, service as a matrix for a catalyst for enabling an enzymatic reaction, limitation or blocking of interfering species, provision of hydrophilicity at the electrochemically reactive surfaces of the sensor interface, service as an interface between host tissue and the implantable device, minimization of the foreign body response or fibrous encapsulation, modulation of host tissue response via drug (or other substance) release, and combinations thereof. When used herein, the terms “membrane” and “matrix” are meant to be interchangeable.

The phrase “membrane system” as used herein is a broad phrase, and is to be given its ordinary and customary meaning to a person of ordinary skill in the art (and is not to be limited to a special or customized meaning), and refers without limitation to a permeable or semi-permeable membrane that can be comprised of two or more domains, layers, or layers within a domain, and is typically constructed of materials of a few microns thickness or more, which is permeable to oxygen and is optionally permeable to, e.g., L-DOPA or another analyte. In examples, the membrane system comprises an immobilized (or covalently coupled) polyphenol oxidase enzyme, which enables a reaction to occur between L-DOPA and oxygen whereby a concentration of L-DOPA can be measured.

The term “noise,” as used herein, is a broad term and is used in its ordinary sense, including, without limitation, a signal detected by the sensor or sensor electronics that is unrelated to analyte concentration and can result in reduced sensor performance. One type of noise has been observed during the few hours (e.g., about 2 to about 24 hours) after sensor insertion. After the first 24 hours, the noise may disappear or diminish, but in some hosts, the noise may last for about three to four days. In some cases, noise can be reduced using predictive modeling, artificial intelligence, and/or algorithmic means. In other cases, noise can be reduced by addressing immune response factors associated with the presence of the implanted sensor, such as using a drug releasing layer with at least one bioactive agent. For example, noise of one or more exemplary biosensors as presently disclosed can be determined and then compared qualitatively or quantitatively. By way of example, by obtaining a raw signal timeseries with a fixed sampling interval (in units of picoampere (pA)), a smoothed version of the raw signal timeseries can be obtained, e.g., by applying a 3rd order lowpass digital Chebyshev Type II filter. Others smoothing algorithms can be used. At each sampling interval, an absolute difference, in units of pA, can be calculated to provide a smoothed timeseries. This smoothed timeseries can be converted into units of mg/dL or mM, (the unit of “noise”), using an L-DOPA sensitivity timeseries, in units of pA/mg/dL or pA/mM, where the L-DOPA sensitivity timeseries is derived by using a mathematical model between the raw signal and reference blood L-DOPA measurements (e.g., obtained from a blood draw). Optionally, the timeseries can be aggregated as desired, e.g., by hour or day. Comparison of corresponding timeseries between different exemplary biosensors with the presently disclosed drug releasing layer and one or more bioactive agents provides for qualitative or quantitative determination of noise improvement.

The term “optional” or “optionally” as used herein is a broad term, and is to be given its ordinary and customary meaning to a person of ordinary skill in the art (and is not to be limited to a special or customized meaning), and, without limitation, means that the subsequently described event or circumstance can or cannot occur, and that the description includes instances where the event or circumstance occurs and instances where it does not.

The term “potentiostat,” as used herein, is a broad term, and is to be given its ordinary and customary meaning to a person of ordinary skill in the art (and is not to be limited to a special or customized meaning), and refers without limitation to an electrical system that applies a potential between the working and reference electrodes of a two- or three-electrode cell at a preset value and measures the current flow through the working electrode. The potentiostat forces whatever current is necessary to flow between the working and counter electrodes to keep the desired potential, as long as the needed cell voltage and current do not exceed the compliance limits of the potentiostat.

The term “proximal” as used herein is a broad term, and is to be given its ordinary and customary meaning to a person of ordinary skill in the art (and is not to be limited to a special or customized meaning), and refers without limitation to the spatial relationship between various elements in comparison to a particular point of reference. For example, some examples of a device include a membrane system having a biointerface layer and an enzyme layer. If the sensor is deemed to be the point of reference and the enzyme layer is positioned nearer to the sensor than the biointerface layer, then the enzyme layer is more proximal to the sensor than the biointerface layer.

The phrase and term “processor module” and “microprocessor” as used herein are each a broad phrase and term, and are to be given their ordinary and customary meaning to a person of ordinary skill in the art (and are not to be limited to a special or customized meaning), and refer without limitation to a computer system, state machine, processor, or the like designed to perform arithmetic or logic operations using logic circuitry that responds to and processes the basic instructions that drive a computer.

The phrase “sensing membrane” as used herein is a broad phrase, and is to be given its ordinary and customary meaning to a person of ordinary skill in the art (and is not to be limited to a special or customized meaning), and refers without limitation to a permeable or semi-permeable membrane that can comprise one or more domains, layers, or layers within domains and that is constructed of materials having a thickness of a few microns or more, and that are permeable to reactants and/or co-reactants employed in determining the analyte of interest. As an example, a sensing membrane can comprise an immobilized polyphenol oxidase enzyme, which catalyzes an electrochemical reaction with L-DOPA and oxygen to permit measurement of a concentration of L-DOPA.

During general operation of the analyte measuring device, biosensor, sensor, sensing region, sensing portion, or sensing mechanism, a biological sample, for example, blood or interstitial fluid, or a component thereof contacts, either directly, or after passage through one or more membranes, an enzyme, for example, polyphenol oxidase, or a protein, for example, one or more periplasmic binding protein (PBP) or mutant or fusion protein thereof having one or more analyte binding regions, each region capable of specifically and reversibly binding to at least one analyte. The interaction of the biological sample or component thereof with the analyte measuring device, biosensor, sensor, sensing region, sensing portion, or sensing mechanism results in transduction of a signal that permits a qualitative, semi-qualitative, quantitative, or semi-qualitative determination of the analyte level, for example, L-DOPA, in the biological sample.

In examples, the sensing region or sensing portion can comprise at least a portion of a conductive substrate or at least a portion of a conductive surface, for example, a wire or conductive trace or a substantially planar substrate including substantially planar trace(s), and a membrane. In examples, the sensing region or sensing portion can comprise a non-conductive body, a working electrode, a reference electrode, and a counter electrode (optional), forming an electrochemically reactive surface at one location on the body and an electronic connection at another location on the body, and a sensing membrane affixed to the body and covering the electrochemically reactive surface. In some examples, the sensing membrane further comprises an enzyme domain, for example, an enzyme layer, and an electrolyte phase, for example, a free-flowing liquid phase comprising an electrolyte-containing fluid described further below. The terms are broad enough to include the entire device, or only the sensing portion thereof (or something in between).

In another example, the sensing region can comprise one or more periplasmic binding protein (PBP) or mutant or fusion protein thereof having one or more analyte binding regions, each region capable of specifically and reversibly binding to at least one analyte. Mutations of the PBP can contribute to or alter one or more of the binding constants, extended stability of the protein, including thermal stability, to bind the protein to a special encapsulation matrix, membrane or polymer, or to attach a detectable reporter group or “label” to indicate a change in the binding region. Specific examples of changes in the binding region include, but are not limited to, hydrophobic/hydrophilic environmental changes, three-dimensional conformational changes, changes in the orientation of amino acid side chains in the binding region of proteins, and redox states of the binding region. Such changes to the binding region provide for transduction of a detectable signal corresponding to the one or more analytes present in the biological fluid.

In examples, the sensing region determines the selectivity among one or more analytes, so that only the analyte which has to be measured leads to (transduces) a detectable signal. The selection may be based on any chemical or physical recognition of the analyte by the sensing region, where the chemical composition of the analyte is unchanged, or in which the sensing region causes or catalyzes a reaction of the analyte that changes the chemical composition of the analyte.

The sensing region transduces the recognition of analytes into a semi-quantitative or quantitative signal. Thus, “transducing” or “transduction” and their grammatical equivalents as are used herein encompasses optical, electrochemical, acoustical/mechanical, or colorimetrical technologies and methods. Electrochemical properties include current and/or voltage, capacitance, and potential. Optical properties include absorbance, fluorescence/phosphorescence, wavelength shift, phase modulation, bio/chemiluminescence, reflectance, light scattering, and refractive index.

The term “sensitivity” as used herein is a broad term, and is to be given its ordinary and customary meaning to a person of ordinary skill in the art (and is not to be limited to a special or customized meaning), and refers without limitation to an amount of signal (e.g., in the form of electrical current and/or voltage) produced by a predetermined amount (unit) of the measured analyte. For example, in examples, a sensor has a sensitivity (or slope) of from about 1 to about 100 nanoamps (e.g., about 20 nA) of current for every 5 μM (98.6 μg/dL of L-DOPA analyte.

### Mutant Enzymes

Electrochemical sensing technology, in examples, utilizes electrodes to apply potential to a system to facilitate a desired chemical reaction. To accomplish this in a biological setting, enzymes are needed to react with analytes in a bodily fluid (e.g., blood, plasma, etc.). These enzymes, however, require sufficient specificity for the analyte to avoid improper signals from the sensor.

As discussed previously herein, L-DOPA sensors are currently under development to ensure that Parkinson's patients have adequate levels of L-DOPA in their systems. These sensors require an enzyme with high activity solely on the target analyte L-DOPA. Existing commercial enzymes, however, also have off-target activity on tyrosine, which is a related and abundant molecule found in humans, thereby precluding the successful construction of a specific L-DOPA sensor.

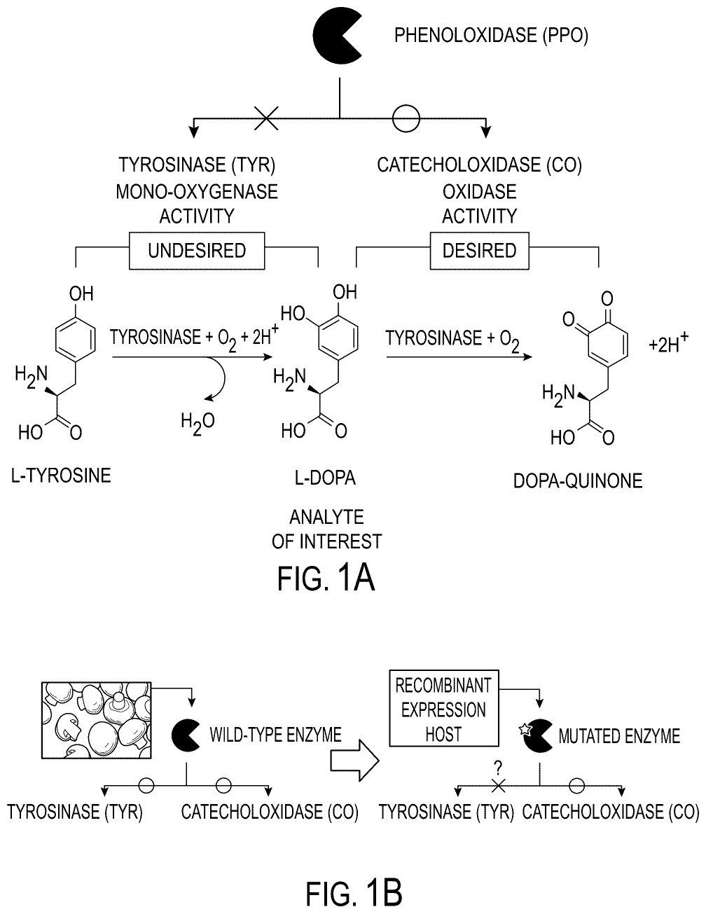

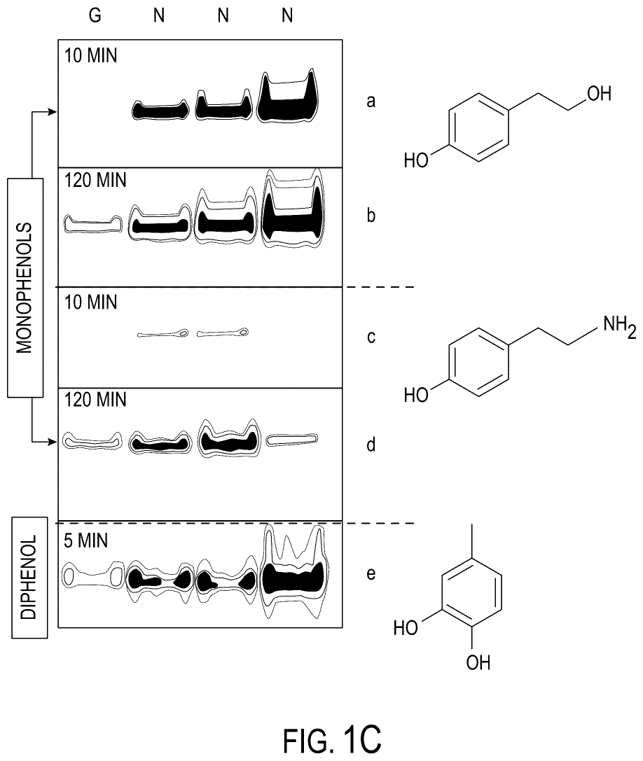

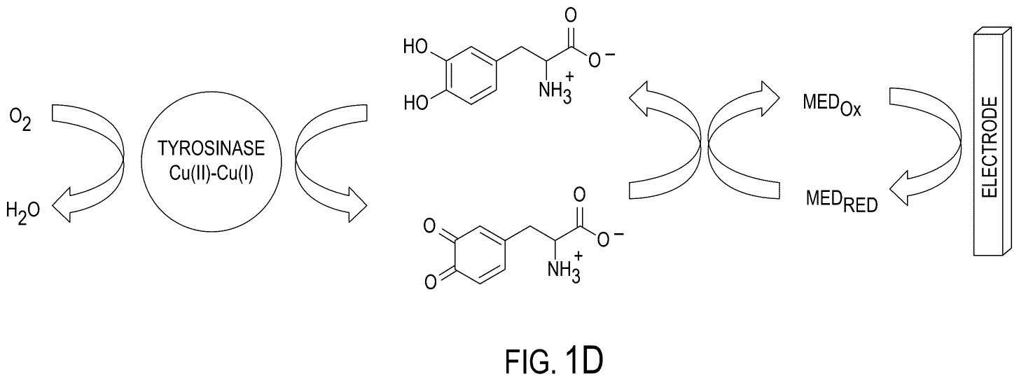

The mutant enzyme described herein, and devices and methods of using the same, provides reduced monophenol activity, thereby resulting in diphenol specificity, to aid in the detection of certain analytes (e.g., catechols such as L-DOPA, dopamine, serotonin, etc.). As can be seen in FIG. 1, for example, when diphenol activity is favored, L-DOPA is detected without interference from L-Tyrosine, which is identified with L-DOPA when monophenol activity is robust.

In examples, an enzyme or a mutated enzyme capable of L-DOPA specificity is provided. In examples, at least one enzyme is selected from a tyrosinase; a mutated tyrosinase with specificity towards l-3,4-dihydroxyphenylalanine; a dihydroxyphenylalanine 4,5-dioxygenase; a mutated dihydroxyphenylalanine 4,5-dioxygenase with specificity towards l-3,4-dihydroxyphenylalanine; and combinations thereof; a synthase enzyme with specificity towards l-3,4-dihydroxyphenylalanine; a mutated synthase enzyme with specificity towards l-3,4-dihydroxyphenylalanine; a 3,4-dihydroxyphenyl-acetaldehyde synthase; a mutated 3,4-dihydroxyphenyl-acetaldehyde synthase with specificity towards l-3,4-dihydroxyphenylalanine; a dioxygenase enzyme with specificity towards l-3,4-dihydroxyphenylalanine; and a mutated dioxygenase enzyme with specificity towards l-3,4-dihydroxyphenylalanine.

A mutated enzyme capable of l-3,4-dihydroxyphenylalanine specificity can be prepared using a recombinant expression host to genetically engineer and express the mutant enzyme. In some examples, the recombinant expression host is a prokaryotic host (e.g., bacteria). In some examples, the recombinant expression host is a bacterium (e.g., Escherichia coli, Bacillus subtilis, Bacillus licheniformis, etc.). In other examples, the recombinant expression host is a eukaryotic host (e.g., fungus, animal, etc.). In some examples, the recombinant expression host is an animal cell. In some examples, the recombinant expression host is insect-derived (e.g., baculovirus-insect cell system). In some examples, the recombinant expression host is a mammalian cell (e.g., Chinese Hamster Ovary). In other examples, the recombinant expression host is a fungus (e.g., yeast). In examples, the recombinant expression host is a yeast (e.g., Saccharomyces cerevisiae, Pichia pastoris, etc.).

Further, in certain examples, the mutant enzyme is derived from a plant, a fungus, or a bacterium. In examples, the mutant enzyme is derived from a fungus (e.g., Agaricus bisporus). In some examples, depending on the source of the PPO enzyme, the mutant PPO enzyme is derived from PPO2 or PPO3.

Polyphenol oxidases use a combination of water, copper, oxygen, and active site residues to achieve substrate specificity and enzymatic function. Copper is held in place with histamine residues, and without intending to be bound by theory, the presence and placement of active site water is conjectured to determine substrate specificity coordinated with a highly conserved glutamine and less conserved asparagine. Enzymes that lack this water-binding asparagine appear to have reduced monophenol activity. As such, it has surprisingly been found that replacing an asparagine with a glycine at a particular location in the PPO enzyme's active site provides the desired diphenol specificity.

The effects of the mutation on respective monophenol and diphenol activity are shown in FIG. 1C. In examples, the mutant PPO enzyme has at least 85% identity to amino acids 1-392 of SEQ ID NO: 1, wherein the mutant PPO enzyme comprises a substitution in a polypeptide chain of a wild-type PPO enzyme active site. In examples, the substitution occurs at amino acid residue position 260 of SEQ ID NO: 1. In further examples, the mutant PPO enzyme has at least 90% identity to amino acids 1-392 of SEQ ID NO: 1. In other examples, the mutant PPO enzyme has at least 95% identity to amino acids 1-392 of SEQ ID NO: 1. In further examples, the mutant PPO enzyme has at least 97% identity to amino acids 1-392 of SEQ ID NO: 1. In some examples, the mutant PPO enzyme has at least 99% identity to amino acids 1-392 of SEQ ID NO: 1.

In examples, PPO enzyme (tyrosinase) enzymes, synthase enzymes (e.g., 1,4-dihydroxyphenylalanine synthase) and dioxygenase enzymes are mutated to provide for one or more of L-DOPA selectivity, e.g., over a 0.1-30 micromolar concentration range, to provide de-selectivity for interferent species present in ISF, to provide thermal stability, to provide sterilization stability, to provide compatibility with one or more membranes or layers, to provide compatibility with electroactive surfaces/electrodes and/or to provide compatibility with mediators. In examples, multiple mutant enzymes (from the same wild type or from different origins with different mutations) are used, for example, to span a targeted L-DOPA concentration range.

FIG. 1D depicts a sensor construct using (mutant) PPO enzyme (tyrosinase) using a mediated system using the L-DOPA conversion to L-DOPA-quinone at a potential of about −0.1V to about −0.3 mV at electrochemically. Suitable electroactive surfaces include graphene oxide or carbon inks with or without embedded mediator in the electroactive surface. In examples, the (mutant) PPO enzyme is embedded in and/or on a carbon electrode. The detection of L-DOPA in ISF throughout the therapeutic levels of about 1-20 micromolar with this sensor construct is provided.

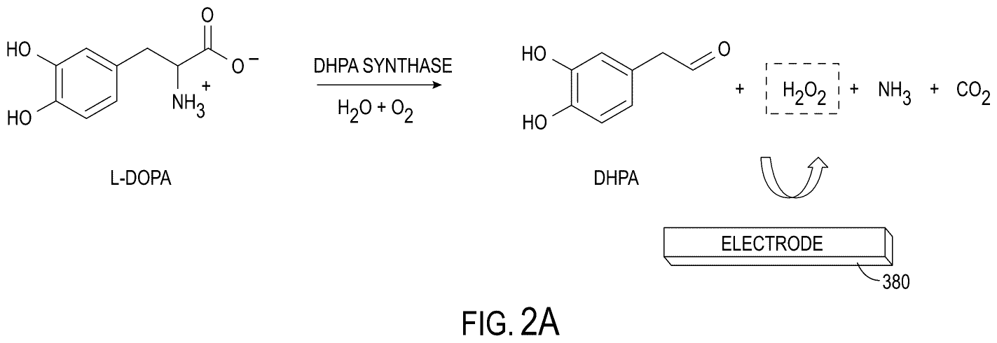

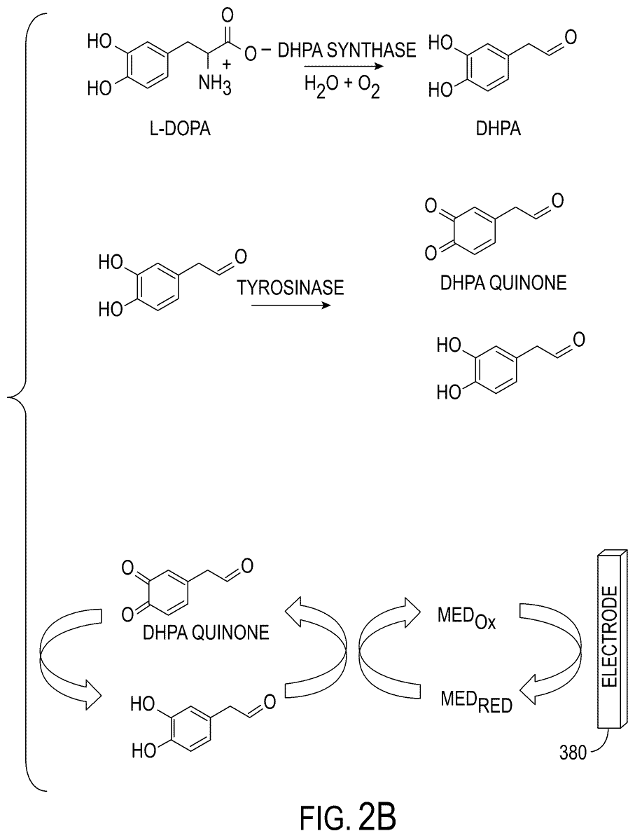

FIG. 2A depicts another sensor construct using (mutant) synthase enzyme (1,4-dihydroxyphenylalanine synthase) where the L-DOPA conversion to DHPA provides hydrogen peroxide, detectable at a potential of about −0.1V to about −0.3 mV at electrode 380. Suitable electroactive surfaces include graphene oxide or carbon inks with or without embedded mediator in the electroactive surface. In examples, the (mutant) synthase enzyme is embedded in and/or on a carbon electrode. The detection of L-DOPA in ISF throughout the therapeutic levels of about 1-20 micromolar using this sensor construct is provided.

FIG. 2B depicts another sensor construct using a cascade approach comprising multiple (mutant) enzymes with electrode 380. In examples, (mutant) synthase enzyme (1,4-dihydroxyphenylalanine synthase (“DHPA synthase”) together with (mutant) tyrosinase is employed. In examples, (mutant) DHPA synthase converts L-DOPA to dihydroxyphenyl acetaldehyde (“DHPAA”, where (mutant) tyrosinase converts the DHPAA to a mediated redox reversible DHPA quinone derivative as shown. In examples, (mutant) synthase enzyme (1,4-dihydroxyphenylalanine synthase (“DHPA synthase”) is combined together with (mutant) tyrosinase in a single membrane or layer. In examples, (mutant) synthase enzyme DHPA synthase and (mutant) tyrosinase are presented in separate membranes or layers. In examples, (mutant) DHPA synthase is arranged more distal from electroactive surface than and (mutant) tyrosinase in separate membranes or layers. One or more additional membranes or layer can be used for interference, resistance, and/or biointerfacing. In examples, the (mutant) synthase includes pyridoxal-5′-phosphate (PLP) as a coenzyme. In examples, the (mutant) synthase enzyme and/or (mutant) tyrosinase is embedded in and/or on a carbon electrode. The detection of L-DOPA in ISF throughout the therapeutic levels of about 1-20 micromolar using this sensor construct is provided.

In examples, electroactive surface fouling by quinone or DHPAA of the aforementioned sensor constructs is reduced or eliminated. In examples, electroactive surface fouling by quinone or DHPAA is reduced or eliminated by controlling local pH environments. In examples, polymer membranes/domains of the sensing membrane include one or more polyelectrolytes configured to create an acidic environment so as to protonate quinone or DHPAA. In examples, the polymer membranes/domains including the one or more polyelectrolytes configured to create an acidic environment are present in the enzyme layer and/or interferent layer. In examples, polyelectrolytes include one or more of polyallylamine, branched polyethyleneimine, poly(acrylamide-co-diallyldimethylammonium chloride), poly(diallyldimethylammonium chloride), poly(2-dimethylamino)ethyl methacrylate) methyl chloride quaternary salt, polyallylamine hydrochloride, branched polyethylenimine, poly(acrylamide-co-diallyldimethylammonium chloride), poly(diallyldimethylammonium chloride), poly(2-dimethylamino)ethyl methacrylate) methyl chloride quaternary salt and blends with one or more polyzwitterion polymers.

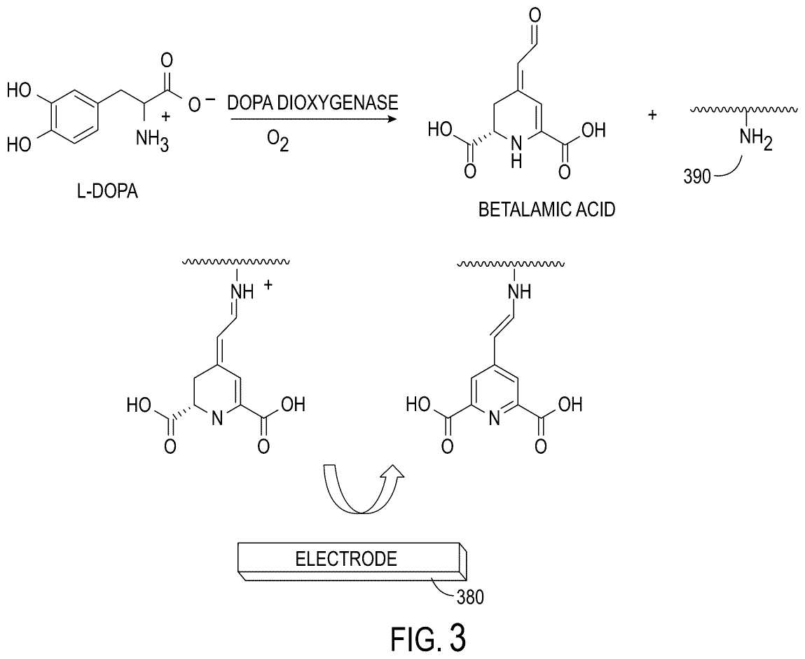

FIG. 3 depicts another sensor construct using a (mutant) dioxygenase enzyme with electrode 380. In examples, DOPA-dioxgenase (“DODA”) is used to provide betalamic acid from L-DOPA. In this construction, at least one membrane or polymer layer 390 comprises free primary or secondary amine groups to interact with the betalamic acid (molar excess, for example) for forming bound betaxanthin imine or enamine, which is electroactive and can be reduced at an electroactive surface to produce a detectable signal corresponding to a L-DOPA concentration. In some examples, the bound betaxanthin or its reduced form can be detected using optical means. In examples, an interference layer is used to attenuate dopamine transport.

### Continuous Monitoring Sensors

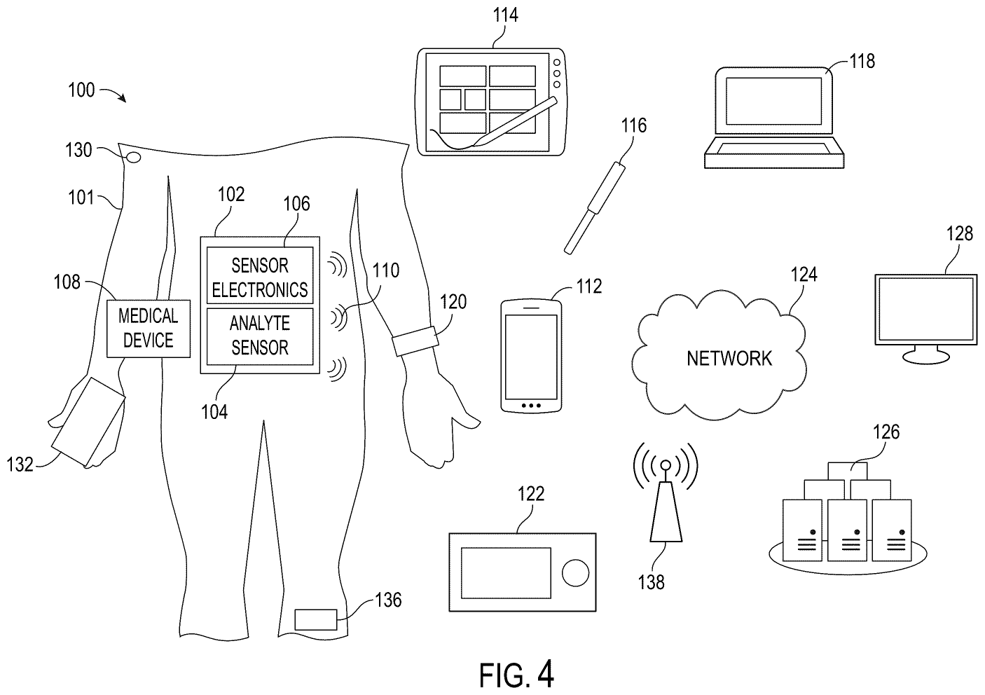

FIG. 4 is an illustration of an example system 100. The system 100 includes an analyte sensor system 102 that is coupled to a host 101. The host 101 may be a human patient. The patient may, for example, be subject to Parkinson's Disease or other health condition for which analyte monitoring may be useful.

The analyte sensor system 102 includes an analyte sensor 104, which may for example be a L-DOPA sensor. In examples, the L-DOPA sensor is any device capable of measuring the concentration of L-DOPA. In examples, the analyte sensor 104 is fully implantable. In another example, the analyte sensor 104 is at least partially wearable on the body with an implantable portion configured for placement under the skin. In yet another example, the analyte sensor 104 is a transcutaneous device (e.g., with a sensor residing under or in the skin of a host). It should be understood that the devices and methods described herein can be applied to any device capable of detecting a concentration of L-DOPA and providing an output signal that represents the concentration of L-DOPA (e.g., as a form of analyte data).

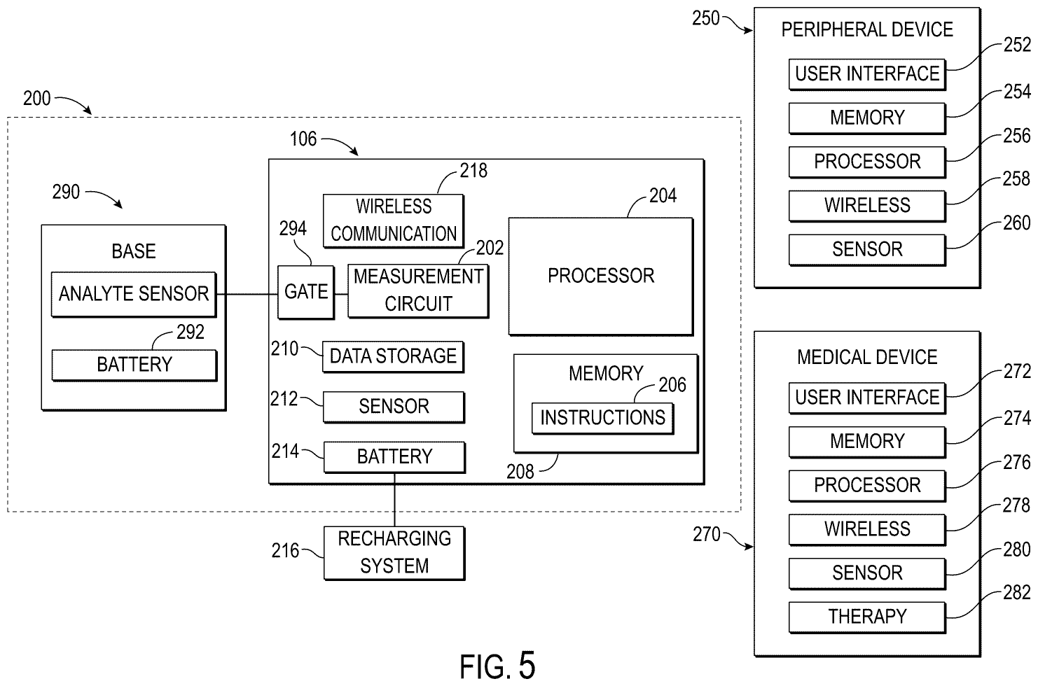

The analyte sensor system 102 also includes sensor electronics 106. In some examples, the analyte sensor 104 and sensor electronics 106 are provided as an integrated package. In other examples, the analyte sensor 104 and sensor electronics 106 are provided as separate components or modules. For example, in one embodiment the analyte sensor system 102 includes a disposable (e.g., single-use) base that may include the analyte sensor 104, a component for attaching the sensor 104 to a host (e.g., an adhesive pad), or a mounting structure configured to receive another component. The system 102 also includes a sensor electronics package, which includes some or all of the sensor electronics 106 shown in FIG. 5. The sensor electronics package may be reusable.

An analyte sensor 104 may use any known method, including invasive, minimally-invasive, or non-invasive sensing techniques (e.g., optically excited fluorescence, microneedle, transdermal monitoring of L-DOPA), to provide a data stream indicative of the concentration of the analyte in a host 101. In some examples, the data stream is a raw data signal, which is converted into a calibrated and/or filtered data stream that is used to provide a useful value of the analyte (e.g., estimated blood L-DOPA concentration level) to a user, such as a patient or a caretaker (e.g., a parent, a relative, a guardian, a teacher, a doctor, a nurse, or any other individual that has an interest in the wellbeing of the host 101).

Analyte sensor 104 may, for example, be a continuous L-DOPA sensor, which may, for example, include a subcutaneous, transdermal (e.g., transcutaneous), or intravascular device. In some embodiments, such a sensor or device may recurrently (e.g., periodically or intermittently) analyze sensor data. The L-DOPA sensor may use any method of L-DOPA measurement, including enzymatic, chemical, physical, electrochemical, spectrophotometric, polarimetric, calorimetric, iontophoretic, radiometric, immunochemical, and the like. In various examples, the analyte sensor system 102 is or includes a continuous L-DOPA monitor sensor.

In some examples, analyte sensor 104 is an implantable L-DOPA sensor, such as described with reference to U.S. Pat. No. 6,001,67 and U.S. Pat. No. 7,778,680, which are incorporated by reference. In some examples, analyte sensor 104 is a transcutaneous L-DOPA sensor, such as described with reference to U.S. Pat. No. 7,497,827, which is incorporated by reference. In some examples, analyte sensor 104 is configured to be implanted in a host vessel or extracorporeally, such as is described in U.S. Pat. No. 7,460,898, U.S. Patent Publication No. US-2008-0119703, U.S. Patent Publication No. US-2008-0108942, and U.S. Pat. No. 7,828,728, all of which are incorporated by reference. In some examples, the continuous L-DOPA sensor includes a transcutaneous sensor such as described in U.S. Pat. No. 6,565,509 to Say et al., which is incorporated by reference. In some examples, analyte sensor 104 is a continuous L-DOPA sensor that includes a subcutaneous sensor such as described with reference to U.S. Pat. No. 6,579,690 to Bonnecaze et al. or U.S. Pat. No. 6,484,46 to Say et al., which are incorporated by reference. In some examples, the continuous L-DOPA sensor includes a refillable subcutaneous sensor such as described with reference to U.S. Pat. No. 6,512,939 to Colvin et al., which is incorporated by reference. In examples, the continuous L-DOPA sensor includes an intravascular sensor such as described with reference to U.S. Pat. No. 6,477,395 to Schulman et al., which is incorporated by reference. In some examples, the continuous L-DOPA sensor includes an intravascular sensor such as described with reference to U.S. Pat. No. 6,424,847 to Mastrototaro et al., which is incorporated by reference.

In some examples, the system 100 also includes a second medical device 108, which may, for example, be a drug delivery device (e.g., L-DOPA pump). In some examples, the medical device 108 includes a sensor, such as another analyte sensor 104, a heart rate sensor, a respiration sensor, a motion sensor (e.g. accelerometer), posture sensor (e.g. 3-axis accelerometer), acoustic sensor (e.g. to capture ambient sound or sounds inside the body). In some examples, medical device 108 may be wearable, e.g., on a watch, glasses, contact lens, patch, wristband, ankle band, or other wearable item, or may be incorporated into a handheld device (e.g., a smartphone). In some examples, the medical device 108 may include a multi-sensor patch that may, for example, detect one or more of an analyte level (e.g., L-DOPA, glucose, lactate, insulin or other substance), heart rate, respiration (e.g., using impedance), activity (e.g., using an accelerometer), posture (e.g., using an accelerometer), galvanic skin response, tissue fluid levels (e.g., using impedance or pressure).

The analyte sensor system 102 may communicate with the second medical device 108 via a wired connection, or via a wireless communication signal 110. For example, the analyte sensor system 102 may be configured to communicate using via radio frequency (e.g., Bluetooth, Medical Implant Communication System (MICS), Wi-Fi, NFC, RFID, Zigbee, Z-Wave or other communication protocols), optically (e.g., infrared), sonically (e.g., ultrasonic), or a cellular protocol (e.g., CDMA (Code Division Multiple Access) or GSM (Global System for Mobiles)), or via a wired connection (e.g., serial, parallel, etc.).

The system 100 may also include a wearable sensor 130, which may include a sensor circuit (e.g., a sensor circuit configured to detect an L-DOPA concentration or other concentration of an analyte) and a communication circuit, which may, for example, be a near field communication (NFC) circuit. In some examples, information from the wearable sensor 130 may be retrieved from the wearable sensor 130 using a user device 132 such as a smart phone that is configured to communicate with the wearable sensor 130 via NFC when the user device 132 is placed near the wearable sensor 130 (e.g., swiping the user device 132 over the sensor 130 retrieves sensor data from the wearable sensor 130 using NFC). The use of NFC communication may reduce power consumption by the wearable sensor 130, which may reduce the size of a power source (e.g., battery or capacitor) in the wearable sensor 130 or extend the usable life of the power source. In some examples, the wearable sensor 130 may be wearable on an upper arm as shown. In some examples, a wearable sensor 130 may additionally or alternatively be on the upper torso of the patient (e.g., over the heart or over a lung), which may, for example, facilitate detecting heart rate, respiration, or posture. A wearable sensor 136 may also be on the lower body (e.g., on a leg).

In some examples, an array or network of sensors may be associated with the patient. For example, one or more of the analyte sensor system 102, medical device 108, wearable device 120 such as a watch, and an additional wearable sensor 130 may communicate with one another via wired or wireless (e.g., Bluetooth, MICS, NFC or any of the other options described above,) communication. The additional wearable sensor 130 may be any of the examples described above with respect to medical device 108. The analyte sensor system 102, medical device 108, and additional sensor 130 on the host 101 are provided for the purpose of illustration and description and are not necessarily drawn to scale.

The system 100 may also include one or more peripheral devices, such as a hand-held smart device (e.g., smartphone) 112, tablet 114, smart pen 116 (e.g., L-DOPA delivery pen with processing and communication capability), computer 118, a wearable device 120 such as a watch, or peripheral medical device 122 (which may be a proprietary device such as a proprietary user device available from DexCom), any of which may communicate with the analyte sensor system 102 via a wireless communication signal 110, and may also communicate over a network 124 with a server system (e.g., remote data center) 126 or with a remote terminal 128 to facilitate communication with a remote user (not shown) such as a technical support staff member or a clinician.

The wearable device 120 may include an activity sensor, a heart rate monitor (e.g., light-based sensor or electrode-based sensor), a respiration sensor (e.g., acoustic- or electrode-based), a location sensor (e.g., GPS), or other sensors.

The system 100 may also include a wireless access point (WAP) 138 that may be used to communicatively couple one or more of analyte sensor system 102, network 124, server system 126, medical device 108 or any of the peripheral devices described above. For example, WAP 138 may provide Wi-Fi and/or cellular connectivity within system 100. Other communication protocols (e.g., Near Field Communication (NFC) or Bluetooth) may also be used among devices of the system 100. In some examples, the server system 126 may be used to collect analyte data from analyte sensor system 102 and/or the plurality of other devices, and to perform analytics on collected data, generate or apply universal or individualized models for L-DOPA levels, and communicate such analytics, models, or information based thereon back to one or more of the devices in the system 100.

FIG. 5 is a schematic illustration of various example electronic components that may be part of a medical device system 200. In examples, the system 200 may include sensor electronics 106 and a base 290. While a specific example of division of components between the base 290 and sensor electronics 106 is shown, it is understood that some examples may include additional components in the base 290 or in the sensor electronics 106, and that some of the components (e.g., a battery or supercapacitor) that are shown in the sensor electronics 106 may be alternatively or additionally (e.g., redundantly) provided in the base 290.

In examples, the base 290 may include the analyte sensor 104 and a battery 292. In some examples, the base 290 may be replaceable, and the sensor electronics 106 may include a debouncing circuit (e.g., gate with hysteresis or delay) to avoid, for example, recurrent execution of a power-up or power down process when a battery is repeatedly connected and disconnected or avoid processing of noise signal associated with removal or replacement of a battery.

The sensor electronics 106 may include electronics components that are configured to process sensor information, such as sensor data, and generate transformed sensor data and displayable sensor information. The sensor electronics 106 may, for example, include electronic circuitry associated with measuring, processing, storing, or communicating continuous analyte sensor data, including prospective algorithms associated with processing and calibration of the sensor data. The sensor electronics 106 may include hardware, firmware, and/or software that enables measurement of levels of the analyte via an L-DOPA sensor. Electronic components may be affixed to a printed circuit board (PCB), or the like, and can take a variety of forms. For example, the electronic components may take the form of an integrated circuit (IC), such as an Application-Specific Integrated Circuit (ASIC), a microcontroller, and/or a processor.

As shown in FIG. 5, the sensor electronics 106 may include a measurement circuit 202 (e.g., potentiostat), which may be coupled to the analyte sensor 104 and configured to recurrently obtain analyte sensor readings using the analyte sensor 104, for example by continuously or recurrently measuring a current flow indicative of concentration of an analyte. The sensor electronics 106 may include a gate circuit 294, which may be used to gate the connection between the measurement circuit 202 and the analyte sensor 104. In examples, the analyte sensor 104 accumulates charge over an accumulation period, and the gate circuit 294 is opened so that the measurement circuit 202 can measure the accumulated charge. Gating the analyte sensor 104 may improve the performance of the sensor system 102 by creating a larger signal to noise or interference ratio (e.g., because charge accumulates from an analyte reaction, but sources of interference do not accumulate, or accumulate less than the charge from the analyte reaction). The sensor electronics 106 may also include a processor 204, which may retrieve instructions 206 from memory 208 and execute the instructions 206 to determine control application of bias potentials to the analyte sensor 104 via the potentiostat, interpret signals from the sensor 104, or compensate for environmental factors. The processor 204 may also save information in data storage memory 210 or retrieve information from data storage memory 210. In various examples, data storage memory 210 may be integrated with memory 208, or may be a separate memory circuit, such as a non-volatile memory circuit (e.g., flash RAM). Examples of systems and methods for processing sensor analyte data are described in more detail herein and in U.S. Pat. Nos. 7,310,544 and 6,931,327.

The sensor electronics 106 may also include a sensor 212, which may be coupled to the processor 204. The sensor 212 may be a temperature sensor, accelerometer, or another suitable sensor. The sensor electronics 106 may also include a power source such as a capacitor or battery 214, which may be integrated into the sensor electronics 106, or may be removable, or part of a separate electronics package. The battery 214 (or other power storage component, e.g., capacitor) may optionally be rechargeable via a wired or wireless (e.g., inductive or ultrasound) recharging system 216. The recharging system 216 may harvest energy or may receive energy from an external source or on-board source. In various examples, the recharge circuit may include a triboelectric charging circuit, a piezoelectric charging circuit, an RF charging circuit, a light charging circuit, an ultrasonic charging circuit, a heat charging circuit, a heat harvesting circuit, or a circuit that harvests energy from the communication circuit. In some examples, the recharging circuit may recharge the rechargeable battery using power supplied from a replaceable battery (e.g., a battery supplied with a base component).

The sensor electronics 106 may also include one or more supercapacitors in the sensor electronics package (as shown), or in the base 290. For example, the supercapacitor may allow energy to be drawn from the battery 214 in a highly consistent manner to extend the life of the battery 214. The battery 214 may recharge the supercapacitor after the supercapacitor delivers energy to the communication circuit or to the processor 204, so that the supercapacitor is prepared for delivery of energy during a subsequent high-load period. In some examples, the supercapacitor may be configured in parallel with the battery 214. A device may be configured to preferentially draw energy from the supercapacitor, as opposed to the battery 214. In some examples, a supercapacitor may be configured to receive energy from a rechargeable battery for short-term storage and transfer energy to the rechargeable battery for long-term storage.

The supercapacitor may extend an operational life of the battery 214 by reducing the strain on the battery 214 during the high-load period. In some examples, a supercapacitor removes at least 10% of the strain off the battery during high-load events. In some examples, a supercapacitor removes at least 20% of the strain off the battery during high-load events. In some examples, a supercapacitor removes at least 30% of the strain off the battery during high-load events. In some examples, a supercapacitor removes at least 50% of the strain off the battery during high-load events.

The sensor electronics 106 may also include a wireless communication circuit 218, which may for example include a wireless transceiver operatively coupled to an antenna. The wireless communication circuit 218 may be operatively coupled to the processor 204 and may be configured to wirelessly communicate with one or more peripheral devices or other medical devices, such as an L-DOPA pump or smart L-DOPA pen.

A peripheral device 250 may, for example, be a wearable device (e.g., activity monitor), such as a wearable device 120. In other examples, the peripheral device 250 may be a hand-held smart device 112 (e.g., smartphone or other device such as a proprietary handheld device available from Dexcom), a tablet 114, a smart pen 116, or special-purpose computer 118 shown in FIG. 4.

The peripheral device 250 may include a user interface 252, a memory circuit 254, a processor 256, a wireless communication circuit 258, a sensor 260, or any combination thereof. The peripheral device 250 may also include a power source, such as a battery. The peripheral device 250 may not necessarily include all of the components shown in FIG. 5. The user interface 252 may, for example, include a touch-screen interface, a microphone (e.g., to receive voice commands), or a speaker, a vibration circuit, or any combination thereof, which may receive information from a user (e.g., L-DOPA values) or deliver information to the user such as L-DOPA values, L-DOPA trends (e.g., an arrow, graph, or chart), or L-DOPA alerts. The processor 256 may be configured to present information to a user, or receive input from a user, via the user interface 252. The processor 256 may also be configured to store and retrieve information, such as communication information (e.g., pairing information or data center access information), user information, sensor data or trends, or other information in the memory circuit 254. The wireless communication circuit 258 may include a transceiver and antenna configured to communicate via a wireless protocol, such as Bluetooth, MICS, or any of the other options described above. The sensor 260 may, for example, include an accelerometer, a temperature sensor, a location sensor, biometric sensor, or blood L-DOPA sensor, blood pressure sensor, heart rate sensor, respiration sensor, or other physiologic sensor. The peripheral device 250 may, for example, be a hand-held smart device 112 (e.g., smartphone or other device such as a proprietary handheld device available from Dexcom), tablet 114, smart pen 116, watch or other wearable device 120, or computer 118 shown in FIG. 4.

The peripheral device 250 may be configured to receive and display sensor information that may be transmitted by sensor electronics 106 (e.g., in a customized data package that is transmitted to the display devices based on their respective preferences). Sensor information (e.g., blood L-DOPA concentration level) or an alert or notification (e.g., “high L-DOPA level”, “low L-DOPA level” or “fall rate alert” may be communicated via the user interface 252 (e.g., via visual display, sound, or vibration). In some examples, the peripheral device 250 may be configured to display or otherwise communicate the sensor information as it is communicated from the sensor electronics 106 (e.g., in a data package that is transmitted to respective display devices). For example, the peripheral device 250 may transmit data that has been processed (e.g., an estimated concentration of an analyte level that may be determined by processing raw sensor data), so that a device that receives the data may not be required to further process the data to determine usable information (such as the estimated concentration of an analyte level). In other examples, the peripheral device 250 may process or interpret the received information (e.g., to declare an alert based on L-DOPA values or a L-DOPA trend). In various examples, the peripheral device 250 may receive information directly from sensor electronics 106, or over a network (e.g., via a cellular or Wi-Fi network that receives information from the sensor electronics 106 or from a device that is communicatively coupled to the sensor electronics 106).

Referring again to FIG. 5, the medical device 270 may include a user interface 272, a memory circuit 274, a processor 276, a wireless communication circuit 278, a sensor 280, a therapy circuit 282, or any combination thereof. The user interface 272 may, for example, include a touch-screen interface, a microphone, or a speaker, a vibration circuit, or any combination thereof, which may receive information from a user (e.g., L-DOPA values, alert preferences, calibration coding) or deliver information to the user, such as e.g., L-DOPA values, L-DOPA trends (e.g., an arrow, graph, or chart), or L-DOPA alerts. The processor 276 may be configured to present information to a user, or receive input from a user, via the user interface 272. The processor 276 may also be configured to store and retrieve information, such as communication information (e.g., pairing information or data center access information), user information, sensor data or trends, or other information in the memory circuit 274. The wireless communication circuit 278 may include a transceiver and antenna configured communicate via a wireless protocol, such as Bluetooth, Medical Implant Communication System (MICS), Wi-Fi, Zigbee, or a cellular protocol (e.g., CDMA (Code Division Multiple Access) or GSM (Global System for Mobiles)). The sensor 280 may, for example, include an accelerometer, a temperature sensor, a location sensor, biometric sensor, or blood L-DOPA sensor, blood pressure sensor, heart rate sensor, respiration sensor, or other physiologic sensor. The medical device 270 may include two or more sensors (or memories or other components), even though only one sensor 280 is shown in the example in FIG. 5. In various examples, the medical device 270 may be a smart handheld L-DOPA sensor (e.g., blood L-DOPA meter), drug pump (e.g., L-DOPA pump), or other physiologic sensor device, therapy device, or combination thereof. In various examples, the medical device 270 may be the medical device 108, peripheral medical device 122, wearable device 120, wearable sensor 130, or wearable sensor 136 shown in FIG. 4.

In examples where the peripheral medical device 122 or medical device 270 is an L-DOPA pump, the pump and analyte sensor system 102 may be in two-way communication (e.g., so the pump can request a change to an analyte transmission protocol, e.g., request a data point or request data on a more frequent schedule), or the pump and analyte sensor system 102 may communicate using one-way communication (e.g., the pump may receive concentration of an analyte level information from the analyte sensor system). In one-way communication, a L-DOPA value may be incorporated in an advertisement message, which may be encrypted with a previously-shared key. In a two-way communication, a pump may request a value, which the analyte sensor system 102 may share, or obtain and share, in response to the request from the pump, and any or all of these communications may be encrypted using one or more previously-shared keys. An L-DOPA pump may receive and track analyte (e.g., L-DOPA) values transmitted from analyte sensor system 102 using one-way communication to the pump for one or more of a variety of reasons. For example, an L-DOPA pump may suspend or activate L-DOPA administration based on a L-DOPA value being below or above a threshold value.

In some examples, the system 100 shown in FIG. 4 may include two or more peripheral devices that each receives information directly or indirectly from the analyte sensor system 102. Because different display devices provide many different user interfaces, the content of the data packages (e.g., amount, format, and/or type of data to be displayed, alarms, and the like) may be customized (e.g., programmed differently by the manufacturer and/or by an end user) for each particular device. For example, in the embodiment of FIG. 4, a plurality of different peripheral devices may be in direct wireless communication with a sensor electronics module (e.g., such as an on-skin sensor electronics 106 that is physically connected to the continuous analyte sensor 104) during a sensor session to enable a plurality of different types and/or levels of display and/or functionality associated with the displayable sensor information, or, to save battery power in the sensor system 102, one or more specified devices may communicate with the analyte sensor system 102 and relay (i.e., share) information to other devices directly or through a server system (e.g., a network-connected data center) 126.

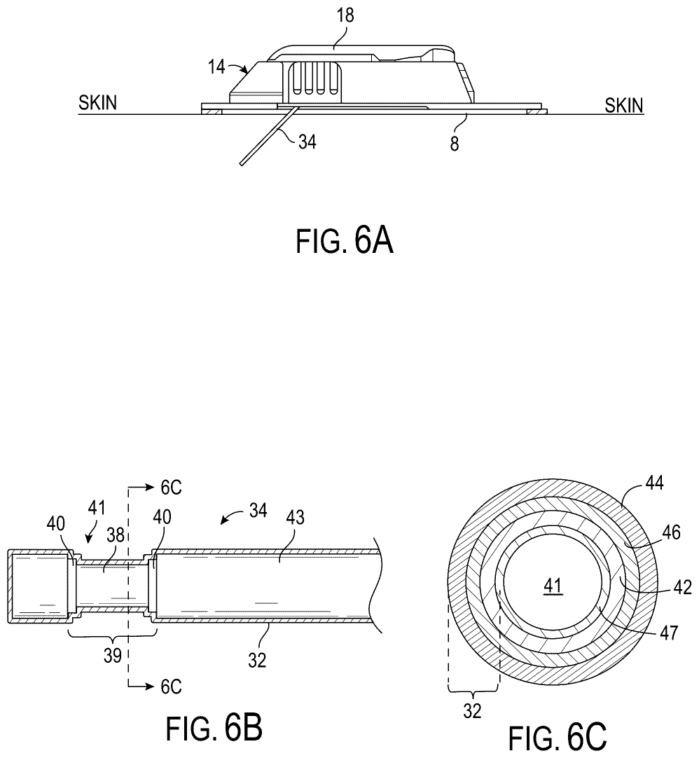

FIG. 6A is a side view of an analyte sensor system, the system mounted on the skin of a host, illustrating an analyte sensor 34 implanted into the host. A mounting unit 14 may be adhered to the host's skin using an adhesive pad 8. The adhesive pad 8 may be formed from an extensible material, which may be removably attached to the skin using an adhesive. The sensor electronics 106 may mechanically couple to the adhesive pad 8.

FIG. 6B is an enlarged view of a distal portion of the analyte sensor 34. The analyte sensor 34 may be adapted for insertion under the host's skin and may be mechanically coupled to the mounting unit 14 and electrically coupled to the sensor electronics (not shown). The example analyte sensor 34 shown in FIG. 6B includes an elongated conductive body 41. The elongated conductive body 41 can include a core with various layers positioned thereon. A first layer 38 that at least partially surrounds the core and includes a working electrode, for example located in window 39. In some examples, the core and the first layer 38 are made of a single material. In some examples, the elongated conductive body 41 is a composite of two conductive materials, or a composite of at least one conductive material and at least one non-conductive material. A membrane system 32 is located over the working electrode and may cover other layers and/or electrodes of the sensor 34, as described herein.

The first layer 38 may be formed of a conductive material. The working electrode (at window 39) is an exposed portion of the surface of the first layer 38. Accordingly, the first layer 38 is formed of a material configured to provide a suitable electroactive surface for the working electrode. Examples of suitable materials include, but are not limited to, platinum, platinum-iridium, gold, palladium, iridium, nitinol, graphite, carbon, a ternary metal oxide composite, a conductive polymer, an alloy, and/or the like.

A second layer 40 surrounds at least a portion of the first layer 38, thereby defining boundaries of the working electrode. In some examples, the second layer 40 serves as an insulator and is formed of an insulating material, such as polyimide, polyurethane, parylene, or any other suitable insulating materials or materials. In some examples, the second layer 40 is configured such that the working electrode of the layer 38 is exposed via the window 39.

In some examples, the sensor 34 further includes a third layer 43 comprising a conductive material. The third layer 43 may comprise a reference electrode. In some examples, the third layer 43, including the reference electrode, is formed of a silver-containing material that is applied onto the second layer 40 e.g., an insulator. The silver-containing material may include various materials and be in various forms such as, for example, Ag/AgCl-polymer pastes, paints, polymer-based conducting mixtures, inks, etc.

The analyte sensor 34 may include two or more electrodes, e.g., a working electrode at the layer 38 and exposed at window 39 and at least one additional electrode, such as a reference electrode of the layer 43. In the example arrangement of FIG. 6B, the reference electrode also functions as a counter electrode, although other arrangements can include a separate counter electrode. While the analyte sensor 34 may be used with a mounting unit in some examples, in other examples, the analyte sensor 34 may be used with other types of sensor systems. For example, the analyte sensor 34 may be part of a system that includes a battery and sensor in a single package, and may optionally include, for example, a near-field communication (NFC) circuit and/or drug delivery unit.

FIG. 6C is a cross-sectional view through the sensor 34 of FIG. 6B on plane 2-2 illustrating an exemplary membrane system 32. In examples, the membrane system 32 includes a number of domains e.g., layers. In examples, the membrane system 32 may include an enzyme domain 42, a diffusion resistance domain 44, and a bioprotective domain 46 located around the working electrode. In some examples, a diffusion resistance domain and bioprotective domain is included in the membrane system 32, e.g., wherein the functionality of both the diffusion resistance domain and bioprotective domain are incorporated into one domain.

The membrane system 32, in some examples, also includes an electrode layer 47. The electrode layer 47 may be arranged to provide an environment between the surfaces of the working electrode and the reference electrode that facilitates the electrochemical reaction between the electrodes. For example, the electrode layer 47 may include a coating that maintains a layer of water at the electrochemically reactive surfaces of the sensor 34.

In some examples, the sensor 34 may be configured for short-term implantation (e.g., from about 1 to 30 days). However, it is understood that the membrane system 32 can be modified for use in other devices, for example, by including only one or more of the domains, or additional domains. For example, a membrane system may include a plurality of resistance layers, or a plurality of enzyme layers. In some examples, the resistance domain 44 may include a plurality of resistance layers, or the enzyme domain 42 may include a plurality of enzyme layers.

The diffusion resistance domain 44 may include a semipermeable membrane that controls the flux of oxygen and L-DOPA to the underlying enzyme domain 42. As a result, the upper limit of linearity of L-DOPA measurement is extended to a much higher value than that which is achieved without the diffusion resistance domain 44.

In some examples, the membrane system 32 may include a bioprotective domain 46, also referred to as a domain or biointerface domain, comprising a base polymer as described in more detail elsewhere herein. However, the membrane system 32 of some examples can also include a plurality of domains or layers including, for example, an electrode domain, an interference domain, or a cell disruptive domain, such as described in more detail elsewhere herein and in U.S. Pat. Nos. 7,494,465, 8,682,408, and 9,44,199, which are incorporated herein by reference in their entirety.

### Dual Electrode/Dual Analyte Sensor

In examples, the presently disclosed continuous analyte sensor is configured for the detection of levodopa and oxygen. In examples, oxygen detection is used to improve the sensitivity and/or accuracy of the determined levodopa concentration by the continuous analyte sensor. In examples, signals corresponding to oxygen and signals corresponding to levodopa are obtained intermittently, e.g., alternating between applied potentials. In examples, signals corresponding to oxygen and signals corresponding to levodopa are obtained in continuous time intervals, e.g., accumulating signals between changing the applied potential.

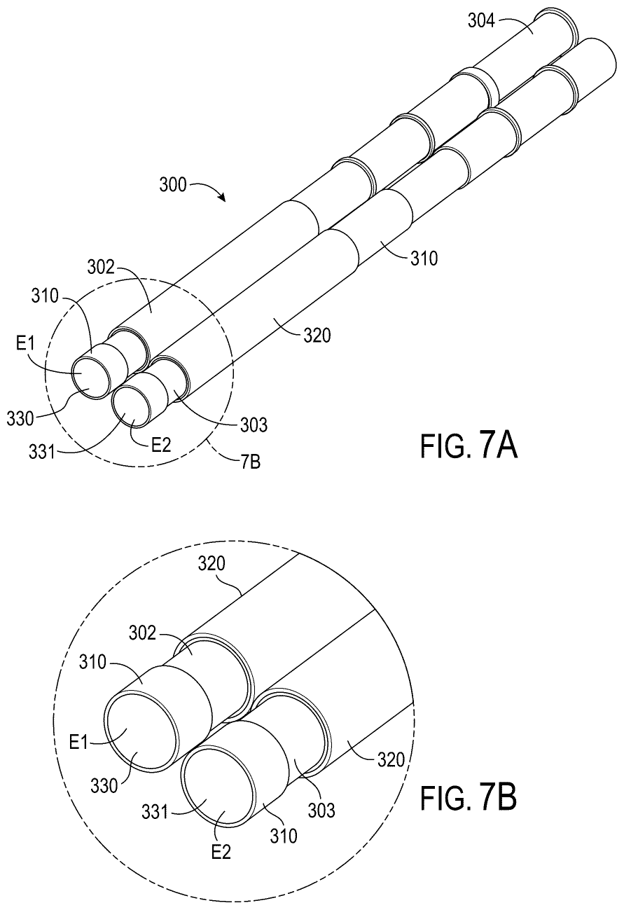

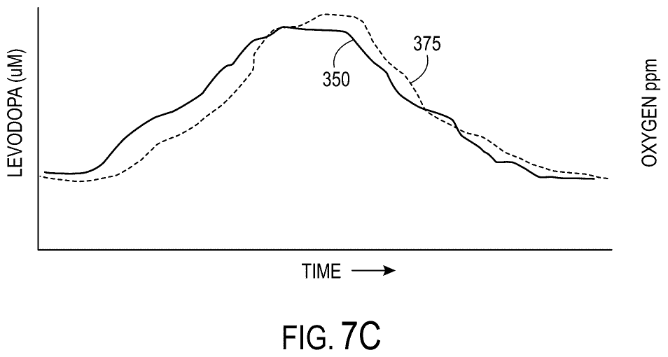

FIG. 7A is a perspective view of the in vivo portion of an example of a multi-electrode sensor system 300 comprising two working electrodes and at least one reference/counter electrode. The sensor 300 comprises first and second elongated bodies E1, E2, each formed of a conductive core or of a core with a conductive layer deposited thereon. In this particular example, a wire-based sensor is shown, however, a planar arrangement is also envisaged. In this particular example, an insulating layer 310, a conductive layer 320 e.g., a reference electrode, and any one of the previously described membranes (not shown) are deposited on top of the elongated bodies E1, E2. The insulating layer 310 separates the conductive layer 320 from the elongated body. The materials selected to form the insulating layer 310 may include any of the insulating materials described elsewhere herein, including polyurethane and polyimide. The materials selected to form the conductive layer 320 may include any of the conductive materials described elsewhere herein, including silver/silver chloride, platinum, gold, etc. Working electrodes 302, 303 are formed by removing portions of the conductive layer 320 and the insulating layer 310, thereby exposing electroactive surface of the elongated bodies E1, E2, respectively. FIG. 7B provides a close perspective view of the distal portion of the elongated bodies E1, E2.

In examples, the two elongated bodies illustrated in FIG. 7A are fabricated to have substantially the same shape and dimensions. In examples, the two elongated bodies illustrated in FIG. 7A are fabricated to have substantially the same shape and dimensions but with one elongated body having no enzyme or a deactivated enzyme, whereas the other elongated body includes an active enzyme with specificity towards l-3,4-dihydroxyphenylalanine (L-DOPA).

In other examples, the two elongated bodies illustrated in FIG. 7A, but with one elongated body having no window and a drug releasing layer, e.g., an anti-inflammatory agent releasing layer, whereas the other elongated body includes a window 39 and sensing membrane with an active enzyme without a drug releasing layer to correct for any lag in signal corresponding to differences in blood and ISF analyte concentrations.

In some examples, the working electrodes of FIG. 7A are fabricated to have the same properties, thereby providing a sensor system capable of providing redundancy of signal measurements or providing unique signals representing two or more different analytes. In other examples, the working electrodes, associated with the elongated bodies E1, E2, may each have one or more characteristics that distinguish each working electrode from the other. For example, In examples, each of the elongated bodies E1, E2 may be different conductive surfaces, so that each working electrode has a different electrochemical property than the other working electrode. In addition, In examples, each of the elongated bodies E1, E2 may be covered with different membrane(s), so that each working electrode has a different membrane property than the other working electrode.

Although not shown in FIGS. 7A-7B, in certain examples, the exposed distal ends 330, 331 of the core portions of the elongated bodies E1, E2 may be covered with an insulating material (e.g., polyurethane or polyimide). In alternative examples, the exposed distal ends 330, 331 of the core portions are covered with any of the previously described membrane system and/or serve as additional or “secondary” working electrode surface area.

Regarding fabrication of the sensor system illustrated in FIG. 7A-7B, In examples, the elongated bodies E1, E2 may be formed as an elongated conductive core, or alternatively as a core (conductive or non-conductive) having at least one conductive material deposited thereon. Next, an insulating layer 310 is deposited onto each of the elongated bodies E1, E2. Thereafter, a conductive layer 320 is deposited over the insulating layer 310. The conductive layer 320 may serve as a reference/counter electrode and may be formed of silver/silver chloride, or any other material that may be used for a reference electrode. In alternative examples, the conductive layer 320 may be formed of a different conductive material, and may be used another working electrode. After these steps, a layer removal process is performed to remove portions of the deposited layers (i.e., the conductive layer 320 and/or the insulating layer 310). Any of the techniques described elsewhere herein (e.g., laser ablation, chemical etching, grit blasting) may be used. In the example illustrated in FIGS. 7A-7B, layers of the conductive layer 320 and the insulating layer 310 are removed to form the working electrodes 302, 303. Although in the example shown, layer removal is performed across the entire cross-sectional perimeter (e.g., circumference) of the deposited layer, it is contemplated that in other examples, layer removal may be performed across a preselected section of the cross-sectional perimeter, instead of across the entire cross-sectional perimeter.

Contacts 304 are used to provide electrical connection between the working electrodes and other components of the sensor system may be formed in a similar manner. As shown, contacts 304 are separated from each other to prevent an electrical connection therebetween. Because the layer removal process is performed on each individual elongated body E1, E2, instead of a single geometrically complicated elongated body, this particular sensor design (i.e., two elongated bodies placed side by side) may provide ease of manufacturing, as compared to the manufacturing processes involved with other multi-electrode systems having other geometries.

After the conductive and insulating layers are deposited onto the elongated body, and after selected portions of the deposited layers have been removed, one or more membranes are applied onto at least a portion of the elongated bodies using the apparatuses and method disclosed herein, either alone or in combination with the apparatuses and method disclosed herein or with other coating apparatuses and methods. In certain examples, any of the aforementioned membrane systems are applied only to the working electrodes, but in other examples any of the aforementioned membrane systems are applied to the entire elongated body. In examples, any of the aforementioned membrane systems are deposited onto the two working electrodes simultaneously while they are placed together (e.g., by bundling), but in other examples, any of the aforementioned membrane systems are deposited onto each individual working electrode first, and the two working electrodes are then placed together.

In examples, the two elongated bodies illustrated in FIG. 7A are fabricated to have substantially the same shape and dimensions but with one elongated body having at least one enzyme with specificity towards l-3,4-dihydroxyphenylalanine (L-DOPA), whereas the other elongated body is an oxygen sensor. In this configuration, the applied potential can be alternated during a suitable frequency to detect L-DOPA and oxygen so that oxygen concentration variability of oxidase enzymes or enzyme systems using L-DOPA and oxygen as substrates can be algorithmically corrected for ISF oxygen levels, if needed.

Enzymatic oxidase biosensors are tunable for a wide dynamic concentration range with the target analyte, however, such enzymes are dependent on oxygen, and thus are sensitive to fluctuations in local oxygen concentrations. Low oxygen concentrations from device compression, foreign body response, exercise, and tissue hypoxia can reduce local oxygen concentration in the subcutaneous tissue. These changes to the local oxygen concentration can affect the oxygen availability to the enzyme and affect sensitivity and/or accuracy.

At 0 to −0.3 V, e.g., at −0.2 V, an exemplary potential for a levodopa biosensor, oxygen is also reduced at this potential and may mask and/or lower resolution of the levodopa signal. Using a single or dual electrode configuration, the levodopa signal can be adjusted based on local oxygen concentration about the implanted portion of the sensor. In examples, using a single wire or single WE planar substrate, potential applied to the WE can be alternated (flipped) at a regular or random time interval, for example between −0.1 V (for generating and detecting levodopa-concentration related signal) and −0.2 V (for generating and detecting oxygen-concentration related signal). In examples, using the levodopa and oxygen signals obtained at different biases, algorithm compensation can be employed to determine a quantitative relationship between CLM sensor sensitivity and oxygen levels. In examples, a Dalziel kinetic model can be used. Other algorithm models can be used. In other examples, a mediated system is used to lower the bias potential and reduce the oxygen signal.

FIG. 7C depicts an exemplary levodopa sensor construct with oxygen concentration adjustment of the detected and determined levodopa concentration. Measured oxygen values 350 are shown superimposed on oxygen-adjusted levodopa levels 375 for a tyrosinase-based CLM construct.

It is to be understood that sensing membranes modified for other sensors, for example, may include fewer or additional layers. In some examples, the membrane system 32 may comprise one electrode layer, one enzyme layer, and two bioprotective layers, but in other examples, the membrane system 32 may comprise one electrode layer, two enzyme layers, and one bioprotective layer. In some examples, the bioprotective layer may be configured to function as the diffusion resistance domain 44 and control the flux of the analyte (e.g., L-DOPA) to the underlying membrane layers. In some examples, the membrane system 32 includes a drug-releasing layer configured to release at least one bioactive agent.

In some examples, one or more domains of the sensing membranes may be formed from materials such as silicone, polytetrafluoroethylene, polyethylene-co-tetrafluoroethylene, polyolefin, polyester, polycarbonate, biostable polytetrafluoroethylene, homopolymers, copolymers, terpolymers of polyurethanes, polypropylene (PP), polyvinylchloride (PVC), polyvinylidene fluoride (PVDF), polybutylene terephthalate (PBT), polymethylmethacrylate (PMMA), polyether ether ketone (PEEK), polyurethanes, cellulosic polymers, poly(ethylene oxide), poly(propylene oxide) and copolymers and blends thereof, polysulfones and block copolymers thereof including, for example, di-block, tri-block, alternating, random and graft copolymers.

In some examples, the sensing membrane can be deposited on the electroactive surfaces of the electrode material using known thin or thick film techniques (for example, spraying, electro-depositing, dipping, or the like). The sensing membrane located over the working electrode does not have to have the same structure as the sensing membrane located over the reference electrode 30; for example, the enzyme domain 42 deposited over the working electrode does not necessarily need to be deposited over the reference or counter electrodes.

Although the examples illustrated in FIGS. 6B-6C and 7A-7C involve circumferentially extending membrane systems, the membranes described herein may be applied to any planar or non-planar surface, for example, the substrate-based sensor structure of U.S. Pat. No. 6,565,509 to Say et al., which is incorporated by reference.

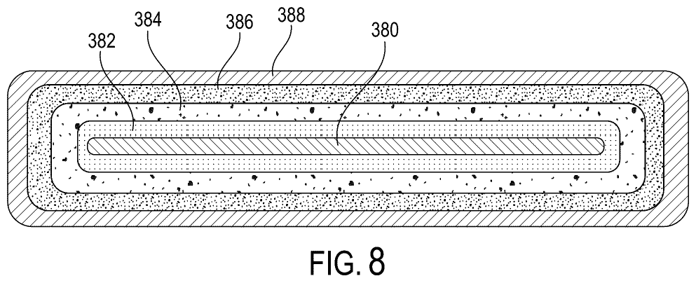

Similar to the circular sensor shown in FIGS. 6B-6C, a planar version shown in FIG. 8 can include electrode 380 with a sensing membrane with multiple layers or domains. For example, the planar version can include an interference domain 382, an enzyme domain 384, and resistance domain 386, in addition to other variations of domains, such as drug releasing membrane 388 as discussed elsewhere herein. As shown, planar version includes sensing membrane surrounding the electrically conductive material or electrode 380, however, the electrically conductive material or electrode 380 can be on one side thereof in other examples.

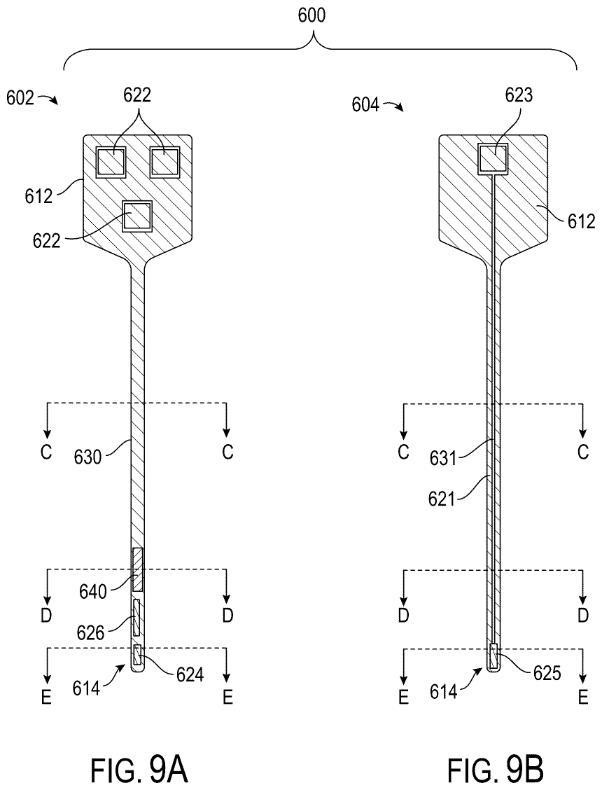

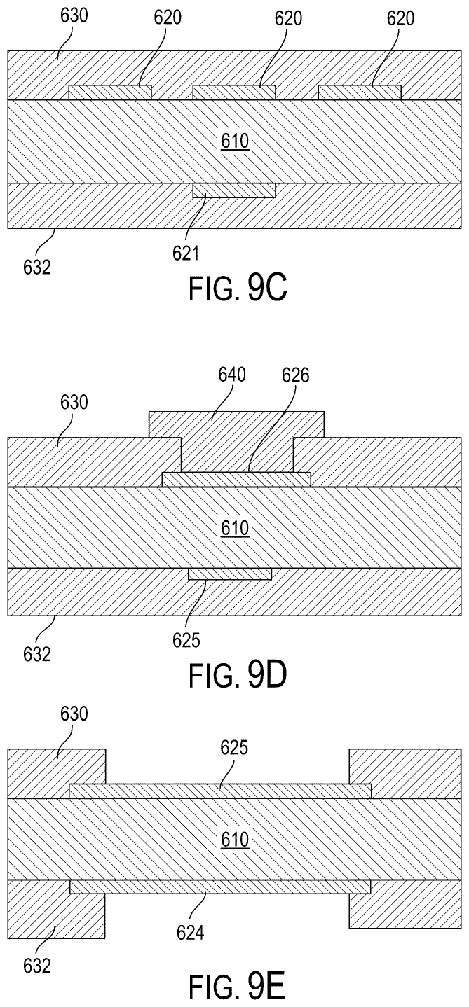

FIGS. 9A to 9B depict an exemplary planar sensor assembly 600, showing top-down drawings of a first side 602 and a second side 604 opposite the first side, in addition to a first end 612 and a second end 614. FIGS. 9C to 9E depict schematic cross-section drawings of the full sensor assembly 600. The sensor assembly 600 can have The sensor assembly 600 can include substrate 610, conductive traces 620, 621, connector pads 622, 623 working electrodes 624, 625, counter electrode 626, insulating layers 630, 632, and reference electrode 640. In sensor assembly 600, a double-sided planar configuration is used. In the sensor assembly 600, a multiple-electrode sensor is shown, with two working electrodes (WE) 624, 625, a counter electrode (CE) 626 and a reference electrode (RE) 640. In sensor assembly 600, the electrodes are co-planar. The sensor assembly 600 is an unconnected variation. In examples, working electrodes (WE) 624, 625 are coated with the sensing membrane with multiple layers or domains as disclosed herein.

In sensor assembly 600, structures can be formed on both sides 602, 604, of the substrate 610. For example, the connector pads 622, 623, can be formed, respectively, on opposing sides 602, 604. This can allow for connection to the sensing electronics from both sides of the sensor assembly 600. Similarly, the conductive traces 620, 621, can be formed on both sides 602, 604, of the sensor assembly 600. On each individual side 602, 604 the conductive traces 620, 621, can be co-planar with each other.

The insulating layers 630, 632, such as a solder mask or other insulating material, can be deposited over the conductive layers including the conductive traces 620, 621. Openings can be formed in the insulating layers 630, 632, to form the working electrodes 624, 625, and the counter electrode 626. An opening can be left for the reference electrode 640. A reference electrode material, such as silver/silver chloride, can be deposited on the designated sensing surface for the reference electrode 640. The insulating material can include epoxy, polyimide, polyurethane, polyethylene, or other materials or combinations of materials.

As illustrated in FIGS. 9A and 9B, the double-sided sensor assembly 600 can include a first working electrode 624, a second working electrode 625, a counter electrode 626, and a reference electrode 640. In some cases, such a double-sided sensor can contain more or less electrodes. For example, a double-sided sensor can include a single working electrode and a reference electrode.

FIGS. 9C to 9E depict cross-sections of the sensor assembly 600. Shown in FIG. 9C is a cross section along line C-C, where the substrate 610 is situated between the two insulating layers 630, 632. The substrate 610 can be, for example, about 50 microns thick. Conductive traces 620, 621, can be seen. On the first side 602, three conductive traces 620 extend along the length of the sensor assembly 600, each connecting to a connector pad 622. The conductive traces 621 on the second side 604 can connect to the connector pad 623.

In FIG. 9D, the cross-section is taken along line D-D. The reference electrode 640 can be seen at this point. In FIG. 9E, the cross-section is taken along line E-E, both working electrodes 624, 625, can be seen on opposing sides 602, 604, of the sensor assembly 600.

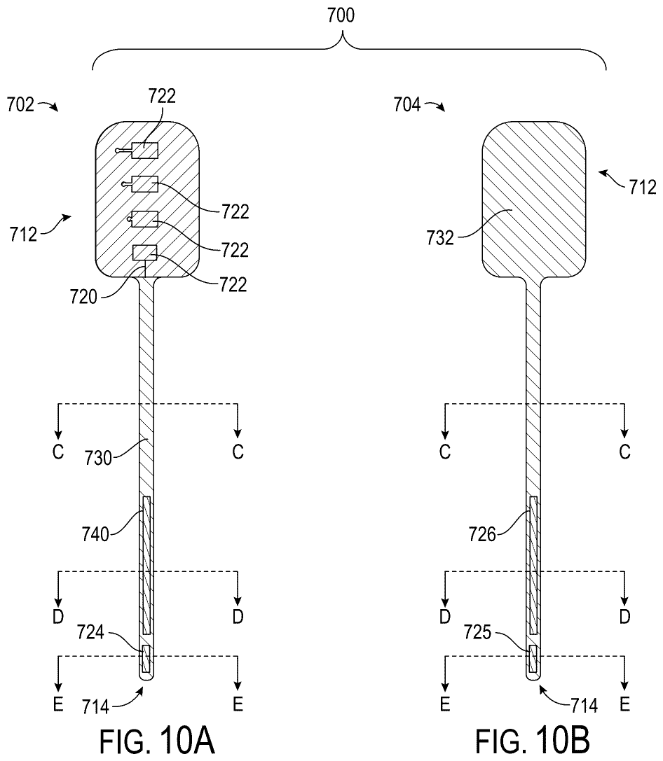

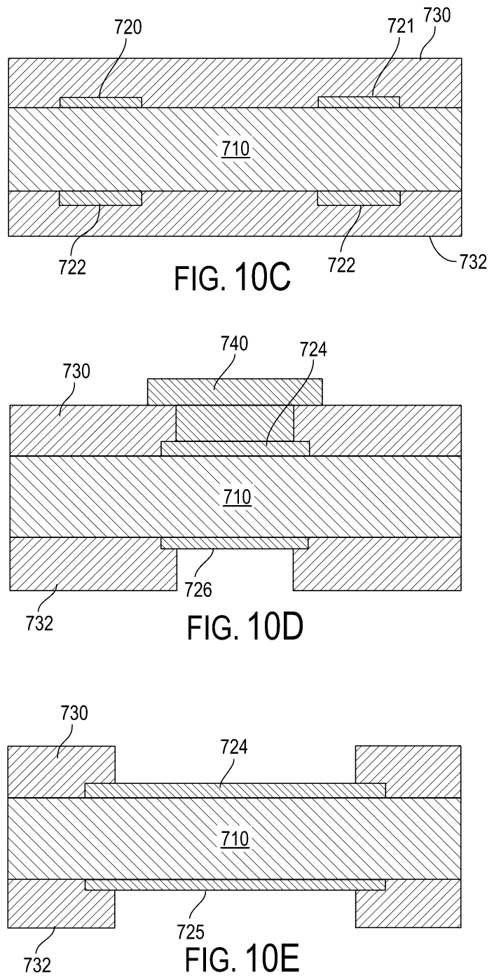

FIGS. 10A-10B illustrate a double-sided co-planar connected analyte sensor assembly 700, in accordance with an example. The sensor assembly 700 can include similar components to those of assembly 600 discussed above, except where otherwise noted.

FIGS. 10A to 10B depict schematic top-down drawings of opposing sides of the assembly 700. FIGS. 10C to 10E depict schematic cross-section drawings along cross-sections taken along C-C, D-D, and E-E, respectively, of the full sensor assembly 700. In some cases, the sensor assembly 700 can include a chamfer end, a rounded end, a flat end, or other appropriate shape. In some cases, the sensor assembly 700 can include a chamfer end, a rounded end, a flat end, or other appropriate shape.

The sensor assembly 700 can have a first side 702 and a second side 704 opposite the first side, in addition to a first end 712 and a second end 714. The sensor assembly 700 can include substrate 710, conductive traces 720, 721, connector pads 722, working electrodes 724, 725, counter electrode 726, insulating layers 730, 732, and reference electrode 740. In sensor assembly 700, a double-sided planar configuration is used. In the sensor assembly 700, a multiple-electrode sensor is shown, with two working electrodes (WE) 724, 725, a counter electrode (CE) 726 and a reference electrode (RE) 740. In sensor assembly 700, the electrodes are co-planar. The assembly 700 is a co-planar, connected variation.

In sensor assembly 700, the substrate 710 is situated between two sides 702, 704, which can each host several co-planar components. For example, co-planar conductive traces 720 can be on the first side 702, and second conductive traces 721 can be on the second side 704. Each side 702, 704, can be covered by an insulating layer 730, 732. The insulating layers 730, 732, can define electrodes 724, 725, 726, and an area for the reference electrode 740.

The assembly 700 can also include via, which can provide for an electrical connection between both sides 702, 704 of the sensor assembly 700. Including vias can allow for connection to the sensing electronics through the connector pads 722 on a single side 702 of the sensor, as well as routing traces to new locations, allowing flexible geometries to be used. The vias can be formed from various conductive materials discussed herein, including carbon, graphitic carbon, Pt, or combinations including Pt and C, Au and C. In some examples, the conductive material forming the vias between sides 702, 704 of the assembly or other assemblies as discussed herein may or may not further include conductive nanoparticles.

Shown in FIGS. 10A to 10E, the assembly 700 can include four connector pads 722 can be on a first side 702, electrically coupled to the electrodes 725, 740, on the second side 704 by vias and traces. In some cases, a WE, RE, and CE can be placed on the opposite side of the sensor assembly 700 to the connector pads 722. In some cases, as shown in assembly 700, a first working electrode 724 and counter electrode 726 can be located on the first side 702 of the sensor, while a second working electrode 725 and a reference electrode 740 can be located on the other side 704 of the sensor. Vias can be used to establish electrical contact between traces and pads on both sides 702, 704 of the sensor assembly 700, since the connector pads 722 for connecting to the sensing electronics, in some examples, are located only on one side. In examples, working electrodes (WE) 724, 725 are coated with the sensing membrane with multiple layers or domains as disclosed herein.

### Electrodes

Examples of electrodes suitable for use in the devices and methods disclosed herein include, for example, platinum and its binary and tertiary alloys, palladium and its binary and tertiary alloys, gold and its binary and tertiary alloys, silver and its binary and tertiary alloys, iridium or indium and its binary and tertiary alloys, nitinol, indium tin oxide, bismuth molybdate (Bi2MoO6), tin sulfide metal oxide (SnS2), boron doped diamond, platinum coated boron doped diamond, conductive graphite and inks therefrom, gold, platinum, pallidum or iridium coated silicon wafers, doped polyaniline, doped poly(3,4-ethylenedioxythio-phene) polystyrene sulfonate (PEDOT:PSS), doped polypyrrole (Ppy), amorphous carbon, carbon nanotubes, graphene metallic nanoparticles, and/or ternary metal oxide composites.

### Sensor/Sensor System

Exemplary sensors are described previously herein. In some examples, the core and first layer can be of a single material (e.g., platinum). In some examples, the elongated conductive body is a composite of at least two materials, such as a composite of two conductive materials, or a composite of at least one conductive material and at least one non-conductive material. In some examples, the elongated conductive body comprises a plurality of layers. In certain examples, there are at least two concentric (e.g., annular) layers, such as a core formed of a first material and a first layer formed of a second material. However, additional layers can be included in some examples. In some examples, the layers are coaxial.

The elongated conductive body may be long and thin, yet flexible and strong. For example, in some examples, the smallest dimension of the elongated conductive body is less than about 0.1 inches, 0.75 inches, 0.5 inches, 0.25 inches, 0.01 inches, 0.004 inches, or 0.002 inches. While the elongated conductive body is shown as having a circular or substantially circular cross-section in some examples, in other examples the cross-section of the elongated conductive body is ovoid, rectangular, triangular, polyhedral, star-shaped, C-shaped, T-shaped, X-shaped, Y-Shaped, irregular, or the like. In examples, a conductive wire electrode is employed as a core. To such a clad electrode, two additional conducting layers may be added (e.g., with intervening insulating layers provided for electrical isolation). The conductive layers can be comprised of any suitable material. In certain examples, it can be desirable to employ a conductive layer comprising conductive particles (i.e., particles of a conductive material) in a polymer or other binder. In other examples, the conductive body can be configured in a linear or planar arrangement, e.g., on a generally flat surface or substrate.

In addition to providing structural support, resiliency and flexibility, in some examples, the core (or a component thereof) provides electrical conduction for an electrical signal from the working electrode to sensor electronics (not shown), which are described elsewhere herein. In some examples, the core comprises a conductive material, such as titanium, stainless steel, tantalum, nitinol, a conductive polymer, and/or the like. However, in other examples, the core is formed from a non-conductive material, such as a non-conductive polymer. In yet other examples, the core comprises a plurality of layers of materials. For example, in examples the core includes an inner core and an outer core. In a further example, the inner core is formed of a first conductive material and the outer core is formed of a second conductive material. For example, in some examples, the first conductive material is stainless steel, titanium, tantalum, a conductive polymer, an alloy, and/or the like, and the second conductive material is conductive material selected to provide electrical conduction between the core and the first layer, and/or to attach the first layer to the core (e.g., if the first layer is formed of a material that does not attach well to the core material). In another example, the core is formed of a non-conductive material (e.g., a non-conductive metal and/or a non-conductive polymer) and the first layer is a conductive material, such as titanium, stainless steel, tantalum, nitinol, a conductive polymer, and/or the like. The core and the first layer can be of a single (or same) material, e.g., platinum. One skilled in the art appreciates that additional configurations are possible.

In some examples, the first layer is formed of a conductive material. The working electrode is an exposed portion of the surface of the first layer. Accordingly, the first layer is formed of a material configured to provide a suitable electroactive surface for the working electrode, a material such as but not limited to platinum, platinum-iridium, gold, palladium, iridium, nitinol, graphite, a ternary metal oxide composite, carbon, a conductive polymer, an alloy and/or the like.

In some example, second layer surrounds a least a portion of the first layer, thereby defining the boundaries of the working electrode. In some examples, the second layer serves as an insulator and is formed of an insulating material, such as polyimide, polyurethane, parylene, or any other known insulating materials, for example, fluorinated polymers, polyethylene terephthalate, polyurethane, polyimide, other nonconducting polymers, or the like. Glass or ceramic materials can also be employed. Other materials suitable for use include surface energy modified coating systems such as are marketed under the trade names AMC18, AMC148, AMC141, and AMC321 by Advanced Materials Components Express of Bellafonte, Pa. In some alternative examples, however, the working electrode may not require a coating of insulator.

In examples, the second layer is disposed on the first layer and configured such that the working electrode is exposed via window. In another example, an elongated conductive body, including the core, the first layer and the second layer, is provided, and the working electrode is exposed (i.e., formed) by removing a portion of the second layer, thereby forming the window through which the electroactive surface of the working electrode (e.g., the exposed surface of the first layer) is exposed. In some examples, the working electrode is exposed by (e.g., window is formed by) removing a portion of the second and (optionally) third layers. Removal of coating materials from one or more layers of elongated conductive body (e.g., to expose the electroactive surface of the working electrode) can be performed by hand, excimer lasing, chemical etching, laser ablation, grit-blasting, or the like.

In some examples, the sensor further comprises a third layer comprising a conductive material. In further examples, the third layer may comprise a reference electrode, which may be formed of a silver-containing material that is applied onto the second layer (e.g., an insulator). The silver-containing material may include any of a variety of materials and be in various forms, such as, Ag/AgCl-polymer pastes, paints, polymer-based conducting mixture, and/or inks that are commercially available, for example. The third layer can be processed using a pasting/dipping/coating step, for example, using a die-metered dip coating process. In one exemplary example, an Ag/AgCl polymer paste is applied to an elongated body by dip-coating the body (e.g., using a meniscus coating technique) and then drawing the body through a die to meter the coating to a precise thickness. In some examples, multiple coating steps are used to build up the coating to a predetermined thickness.

In some examples, the silver grain in the Ag/AgCl solution or paste can have an average particle size corresponding to a maximum particle dimension that is less than about 100 microns, or less than about 50 microns, or less than about 30 microns, or less than about 20 microns, or less than about 10 microns, or less than about 5 microns. The silver chloride grain in the Ag/AgCl solution or paste can have an average particle size corresponding to a maximum particle dimension that is less than about 100 microns, or less than about 80 microns, or less than about 60 microns, or less than about 50 microns, or less than about 20 microns, or less than about 10 microns. The silver grain and the silver chloride grain may be incorporated at a ratio of the silver chloride grain:silver grain of from about 0.01:1 to 2:1 by weight, or from about 0.1:1 to 1:1. The silver grains and the silver chloride grains are then mixed with a carrier (e.g., a polyurethane) to form a solution or paste. In certain examples, the Ag/AgCl component form from about 10% to about 65% by weight of the total Ag/AgCl solution or paste, or from about 20% to about 50%, or from about 23% to about 37%. In some examples, the Ag/AgCl solution or paste has a viscosity (under ambient conditions) that is from about 1 to about 500 centipoise, or from about 10 to about 300 centipoise, of from about 50 to about 150 centipoise.

In examples, the above-exemplified sensor has an overall diameter of not more than about 0.20 inches (about 0.51 mm), more preferably not more than about 0.18 inches (about 0.46 mm), and most preferably not more than about 0.16 inches (0.41 mm). In some examples, the working electrode has a diameter of from about 0.001 inches or less to about 0.10 inches or more, preferably from about 0.002 inches to about 0.008 inches, and more preferably from about 0.004 inches to about 0.005 inches. The length of the window can be from about 0.1 mm (about 0.004 inches) or less to about 2 mm (about 0.78 inches) or more, and preferably from about 0.5 mm (about 0.2 inches) to about 0.75 mm (0.03 inches). In such examples, the exposed surface area of the working electrode is preferably from about 0.000013 in2 (0.0000839 cm2) or less to about 0.0025 in2(0.016129 cm2) or more (assuming a diameter of from about 0.001 inches to about 0.10 inches and a length of from about 0.004 inches to about 0.78 inches). The exposed surface area of the working electrode is selected to produce an analyte signal with a current in the femtoampere range, picoampere range, the nanoampere range, the or the microampere range such as is described in more detail elsewhere herein. However, a current in the picoampere range or less can be dependent upon a variety of factors, for example the electronic circuitry design (e.g., sample rate, current draw, A/D converter bit resolution, etc.), the membrane system (e.g., permeability of the analyte through the membrane system), and the exposed surface area of the working electrode. Accordingly, the exposed electroactive working electrode surface area can be selected to have a value greater than or less than the above-described ranges taking into consideration alterations in the membrane system and/or electronic circuitry. In examples of a L-DOPA sensor, it can be advantageous to minimize the surface area of the working electrode while maximizing the diffusivity of L-DOPA in order to optimize the signal-to-noise ratio while maintaining sensor performance in both high and low L-DOPA concentration ranges.

In some alternative examples, the exposed surface area of the working (and/or other) electrode can be increased by altering the cross-section of the electrode itself. For example, in some examples the cross-section of the working electrode can be defined by a cross, star, cloverleaf, ribbed, dimpled, ridged, irregular, or other non-circular configuration; thus, for any predetermined length of electrode, a specific increased surface area can be achieved (as compared to the area achieved by a circular cross-section). Increasing the surface area of the working electrode can be advantageous in providing an increased signal responsive to the concentration of an analyte, which in turn can be helpful in improving the signal-to-noise ratio, for example.

In some examples, the elongated conductive body further comprises one or more intermediate layers located between the core and the first layer. For example, in some examples, the intermediate layer is an insulator, a conductor, a polymer, and/or an adhesive.

In certain example, the core comprises a non-conductive polymer and the first layer comprises a conductive material. Such a sensor configuration can sometimes provide reduced material costs, in that it replaces a typically expensive material with an inexpensive material. For example, in some examples, the core is formed of a non-conductive polymer, such as, a nylon or polyester filament, string or cord, which can be coated and/or plated with a conductive material, such as platinum, platinum-iridium, gold, palladium, iridium, graphite, carbon, a conductive polymer, and allows or combinations thereof.

In some examples, the sensor also includes a membrane covering at least a portion of the working electrode. Membranes are discussed in detail in greater detail elsewhere herein.

Exemplary sensor configurations may be applied to any planar or non-planar surface, for example. In another example, the sensor system has additional electrodes arranged as one or more concentric substantially ring-shaped electrodes, or rows or arrays of electrodes on a planar or substantially planar substrate.

As discussed herein, in some examples, the membrane system includes a bioprotective domain, also referred to as a cell-impermeable domain or biointerface domain, comprising a surface-modified base polymer as described in more detail elsewhere herein. In some examples, a unitary diffusion resistance domain and bioprotective domain may be included in the membrane system (e.g., wherein the functionality of both domains is incorporated into one domain, i.e., the bioprotective domain). In some examples, the sensor is configured for implantation from about 1 to 30 days). However, it is understood that the membrane system can be modified for use in other devices, for example, by including only one or more of the domains, or additional domains.

In some examples, the membrane system may include an electrode domain. The electrode domain is provided to ensure that an electrochemical reaction occurs between the electroactive surfaces of the working electrode and the reference electrode, and thus the electrode domain may be situated more proximal to the electroactive surfaces than the interference and/or enzyme domain. The electrode domain may include a coating that maintains a layer of water at the electrochemically reactive surfaces of the sensor. In other words, the electrode domain may be present to provide an environment between the surfaces of the working electrode and the reference electrode, which facilitates an electrochemical reaction between the electrodes.

A wide variety of configurations and combinations for the various layers in the membrane system are encompassed by the examples. In various examples, any of the domains described herein may be omitted, altered, substituted for, and/or incorporated together without departing from the spirit of the preferred examples. It is to be understood that sensing membranes modified for other sensors, for example, may include fewer or additional layers. For example, in some examples, the membrane system may comprise one electrode layer, one enzyme layer, and two bioprotective layers, but in other examples, the membrane system may comprise one electrode layer, two enzyme layers, and one bioprotective layer. In some examples, the bioprotective layer may be configured to function as the diffusion resistance domain and control the flux of the analyte (e.g., L-DOPA) to the underlying membrane layers.

In some examples, a sensing membrane comprising one or more domains of polymeric membranes may be formed from materials such as polytetrafluoroethylene, silicone, polyethylene-co-tetrafluoroethylene, polyolefin, polyester, polycarbonate, biostable polytetrafluoroethylene, homopolymers, copolymers, terpolymers of polyurethanes, polypropylene (PP), polyvinylchloride (PVC), polyvinylidene fluoride (PVDF), polybutylene terephthalate (PBT), polymethylmethacrylate (PMMA), polyether ether ketone (PEEK), polyurethanes, cellulosic polymers, poly(ethylene oxide), poly(propylene oxide) and copolymers and blends thereof, polysulfones and block copolymers thereof including, for example, di-block, tri-block, alternating, random and graft copolymers.

In examples, a sensing membrane is disposed over the electroactive surfaces of the continuous transcutaneous analyte sensor and includes one or more domains or layers of a membrane system. In general, the sensing membrane functions to control the flux of a biological fluid there through and/or to protect sensitive regions of the sensor from contamination by the biological fluid, for example. Some conventional electrochemical enzyme-based analyte sensors generally include a sensing membrane that controls the flux of the analyte being measured, protects the electrodes from contamination of the biological fluid, and/or provides an enzyme that catalyzes the reaction of the analyte with a co-factor, for example. See, e.g., U.S. Patent Publication No. 2005-0245799A1 and U.S. Pat. No. 7,497,827, which are incorporated herein by reference in their entirety.

The sensing membranes of the present disclosure can include any membrane configuration suitable for use with any analyte sensor (such as described in more detail above). In general, the sensing membranes of the present disclosure include one or more domains, all or some of which can be adhered to or deposited on the analyte sensor as is appreciated by one skilled in the art. In examples, the sensing membrane generally provides one or more of the following functions: 1) protection of the exposed electrode surface from the biological environment, 2) diffusion resistance (limitation) of the analyte, 3) a catalyst for enabling an enzymatic reaction, 4) limitation or blocking of interfering species, and 5) hydrophilicity at the electrochemically reactive surfaces of the sensor interface, such as described in the above-referenced U.S. patents and patent publications.

### Membrane Systems

In some examples, one or more domains of the membranes are formed from materials such as silicone, polytetrafluoroethylene, polyethylene-co-tetrafluoroethylene, polyolefin, polyester, polycarbonate, biostable polytetrafluoroethylene, homopolymers, copolymers, terpolymers of polyurethanes, polypropylene (PP), polyvinylchloride (PVC), polyvinylidene fluoride (PVDF), polybutylene terephthalate (PBT), polymethylmethacrylate (PMMA), polyether ether ketone (PEEK), polyurethanes, cellulosic polymers, poly(ethylene oxide), poly(propylene oxide) and copolymers and blends thereof, polysulfones and block copolymers thereof including, for example, di-block, tri-block, alternating, random and graft copolymers. U.S. Patent Publication No. 2005-0245799A1, which is incorporated herein by reference in its entirety, describes biointerface and sensing membrane configurations and materials that may be applied to the presently disclosed sensor.

### Electrode Domain

In some examples, the membrane system comprises an optional electrode domain. The electrode domain is provided to ensure that an electrochemical reaction occurs between the electroactive surfaces of the working electrode and the reference electrode, and thus the electrode domain is preferably situated more proximal to the electroactive surfaces than the enzyme domain. Preferably, the electrode domain includes a semipermeable coating that maintains a layer of water at the electrochemically reactive surfaces of the sensor, for example, a humectant in a binder material can be employed as an electrode domain; this allows for the full transport of ions in the aqueous environment. The electrode domain can also assist in stabilizing the operation of the sensor by overcoming electrode start-up and drifting problems caused by inadequate electrolyte. The material that forms the electrode domain can also protect against pH-mediated damage that can result from the formation of a large pH gradient due to the electrochemical activity of the electrodes.

In examples, the electrode domain includes a flexible, water-swellable, hydrogel film having a “dry film” thickness of from about 0.5 micron or less to about 20 microns or more, more preferably from about 0.5, 0.1, 0.15, 0.2, 0.25, 0.3, 0.35, 0.4, 0.45, 0.5, 1, 1.5, 2, 2.5, 3, or 3.5 to about 4, 5, 6, 7, 8, 9, 10, 11, 12, 13, 14, 15, 16, 17, 18, 19, or 19.5 microns, and more preferably from about 2, 2.5 or 3 microns to about 3.5, 4, 4.5, or 5 microns. “Dry film” thickness refers to the thickness of a cured film cast from a coating formulation by standard coating techniques.

In certain examples, the electrode domain is formed of a curable mixture of a urethane polymer and a hydrophilic polymer. Particularly preferred coatings are formed of a polyurethane polymer having carboxylate functional groups and non-ionic hydrophilic polyether segments, wherein the polyurethane polymer is crosslinked with a water soluble carbodiimide (e.g., 1-ethyl-3-(3-dimethylaminopropyl)carbodiimide (EDC))) in the presence of polyvinylpyrrolidone and cured at a moderate temperature of about 50° C.

Preferably, the electrode domain is deposited by spray or dip-coating the electroactive surfaces of the sensor. More preferably, the electrode domain is formed by dip-coating the electroactive surfaces in an electrode solution and curing the domain for a time of from about 15 to about 30 minutes at a temperature of from about 40 to about 55° C. (and can be accomplished under vacuum (e.g., 20 to 30 mmHg)). In examples wherein dip-coating is used to deposit the electrode domain, a preferred insertion rate of from about 1 to about 3 inches per minute, with a preferred dwell time of from about 0.5 to about 2 minutes, and a preferred withdrawal rate of from about 0.25 to about 2 inches per minute provide a functional coating. However, values outside of those set forth above can be acceptable or even desirable in certain examples, for example, dependent upon viscosity and surface tension as is appreciated by one skilled in the art. In examples, the electroactive surfaces of the electrode system are dip-coated one time (one layer) and cured at 50° C. under vacuum for 20 minutes.

Although an independent electrode domain is described herein, in some examples, sufficient hydrophilicity can be provided in the interference domain and/or enzyme domain (the domain adjacent to the electroactive surfaces) so as to provide for the full transport of ions in the aqueous environment (e.g. without a distinct electrode domain).

### Interference Domain

In some examples, an optional interference domain is provided, which generally includes a polymer domain that restricts the flow of one or more interferants. In some examples, the interference domain functions as a molecular sieve that allows analytes and other substances that are to be measured by the electrodes to pass through, while preventing passage of other substances, including interferants such as ascorbate and urea (see U.S. Pat. No. 6,001,67 to Shults). Some known interferants are caffeic acid, dopamine, L-tyrosine, 3-o-methyldopa, L-alpha-methyldopa, homocysteine, carbidopa, cresols (e.g., m-cresol, an insulin preservative), parabens (drug preservatives), and the like.

Several polymer types that can be utilized as a base material for the interference domain include polyurethanes, polymers having pendant ionic groups, and polymers having controlled pore size, for example. In examples, the interference domain includes a thin, hydrophobic membrane that is non-swellable and restricts diffusion of low molecular weight species. The interference domain is permeable to relatively low molecular weight substances but restricts the passage of higher molecular weight substances. Other systems and methods for reducing or eliminating interference species that can be applied to the membrane system of the present disclosure are described in U.S. Pat. No. 7,816,004, U.S. Patent Publication No. 2005-0176136A1, U.S. pat. No. 7,81,195, and U.S. Pat. No. 7,715,893. In some alternative examples, a distinct interference domain is not included.

In examples, the interference domain is deposited onto the electrode domain (or directly onto the electroactive surfaces when a distinct electrode domain is not included) for a domain thickness of from about 0.5 micron or less to about 20 microns or more, more preferably from about 0.5, 0.1, 0.15, 0.2, 0.25, 0.3, 0.35, 0.4, 0.45, 0.5, 1, 1.5, 2, 2.5, 3, or 3.5 to about 4, 5, 6, 7, 8, 9, 10, 11, 12, 13, 14, 15, 16, 17, 18, 19, or 19.5 microns, and more preferably from about 2, 2.5 or 3 microns to about 3.5, 4, 4.5, or 5 microns. Thicker membranes can also be useful, but thinner membranes are generally preferred because they have a lower impact on the rate of diffusion of dopaquinone from the enzyme membrane to the electrodes. Unfortunately, the thin thickness of the interference domains conventionally used can introduce variability in the membrane system processing. For example, if too much or too little interference domain is incorporated within a membrane system, the performance of the membrane can be adversely affected.

### Enzyme Domain

In examples, the membrane system further includes an enzyme domain disposed more distally from the electroactive surfaces than the interference domain (or electrode domain when a distinct interference is not included). In some examples, the enzyme domain is directly deposited onto the electroactive surfaces (when neither an electrode or interference domain is included). In examples, the enzyme domain provides an enzyme to catalyze the reaction of the analyte and its co-reactant, as described in more detail below. Preferably, the enzyme domain includes polyphenol oxidase.

For an enzyme-based electrochemical L-DOPA sensor to perform well, the sensor's response is preferably limited by neither enzyme activity nor co-reactant concentration. Because enzymes, including polyphenol oxidase, are subject to deactivation as a function of time even in ambient conditions, this behavior is compensated for in forming the enzyme domain. Preferably, the enzyme domain is constructed of aqueous dispersions of colloidal polyurethane polymers including the enzyme. However, in alternative examples the enzyme domain is constructed from an oxygen enhancing material, for example, silicone, or fluorocarbon, in order to provide a supply of excess oxygen during transient ischemia. Preferably, the enzyme is immobilized within the domain. See U.S. Pat. No. 7,379,765.

In examples, the enzyme domain is deposited onto the interference domain for a domain thickness of from about 0.5 micron or less to about 20 microns or more, more preferably from about 0.5, 0.1, 0.15, 0.2, 0.25, 0.3, 0.35, 0.4, 0.45, 0.5, 1, 1.5, 2, 2.5, 3, or 3.5 to about 4, 5, 6, 7, 8, 9, 10, 11, 12, 13, 14, 15, 16, 17, 18, 19, or 19.5 microns, and more preferably from about 2, 2.5 or 3 microns to about 3.5, 4, 4.5, or 5 microns. However in some examples, the enzyme domain is deposited onto the electrode domain or directly onto the electroactive surfaces. Preferably, the enzyme domain is deposited by spray or dip coating. More preferably, the enzyme domain is formed by dip-coating the electrode domain into an enzyme domain solution and curing the domain for from about 15 to about 30 minutes at a temperature of from about 40 to about 55° C. (and can be accomplished under vacuum (e.g., 20 to 30 mmHg)). In examples wherein dip-coating is used to deposit the enzyme domain at room temperature, a preferred insertion rate of from about 1 inch per minute to about 3 inches per minute, with a preferred dwell time of from about 0.5 minutes to about 2 minutes, and a preferred withdrawal rate of from about 0.25 inch per minute to about 2 inches per minute provide a functional coating. However, values outside of those set forth above can be acceptable or even desirable in certain examples, for example, dependent upon viscosity and surface tension as is appreciated by one skilled in the art. In examples, the enzyme domain is formed by dip coating two times (namely, forming two layers) in a coating solution and curing at 50° C. under vacuum for 20 minutes. However, in some examples, the enzyme domain can be formed by dip-coating and/or spray-coating one or more layers at a predetermined concentration of the coating solution, insertion rate, dwell time, withdrawal rate, and/or desired thickness.

### Resistance Domain

In examples, the membrane system includes a resistance domain disposed more distal from the electroactive surfaces than the enzyme domain. Although the following description is directed to a resistance domain for an L-DOPA sensor, the resistance domain can be modified for other analytes and co-reactants as well.

The resistance domain includes a semi-permeable membrane that controls the flux of oxygen and L-DOPA to the underlying enzyme domain, preferably rendering oxygen in a non-rate-limiting excess. As a result, the upper limit of linearity of L-DOPA measurement is extended to a much higher value than that which is achieved without the resistance domain. In examples, the resistance domain exhibits an oxygen to L-DOPA permeability ratio such that one-dimensional reactant diffusion is adequate to provide excess oxygen at all reasonable L-DOPA and oxygen concentrations found in the subcutaneous matrix.

In alternative examples, a lower ratio of oxygen-to-L-DOPA can be sufficient to provide excess oxygen by using a high oxygen solubility domain (for example, a silicone or fluorocarbon-based material or domain) to enhance the supply/transport of oxygen to the enzyme domain. If more oxygen is supplied to the enzyme, then more L-DOPA can also be supplied to the enzyme without creating an oxygen rate-limiting excess. In alternative examples, the resistance domain is formed from a silicone composition, such as is described in U.S. Patent Publication No. US 2005/0090607 filed Oct. 28, 2003 and entitled, “SILICONE COMPOSITION FOR BIOCOMPATIBLE MEMBRANE.”

In examples, the presently disclosed CLM sensor includes a resistance domain to control the diffusion of L-DOPA and oxygen to the CLM sensor, fabricated easily and reproducibly from commercially available materials. A suitable resistance domain component is a polyurethane or polyurethaneurea (hereinafter, collectively referred to as “PU”) which may be a thermoplastic polyurethane or polyurethaneurea or blend thereof. Polyurethane is a polymer produced by the condensation reaction of a diisocyanate and a difunctional hydroxyl-containing material. A polyurethaneurea is a polymer produced by the condensation reaction of a diisocyanate and a difunctional amine-containing material. Exemplary diisocyanates include aliphatic diisocyanates containing from about 4 to about 8 methylene units. Diisocyanates containing cycloaliphatic moieties can also be useful in the preparation of the polymer and copolymer components of the membranes of the present disclosure.

In examples, a PU polymer is provided with a hard segment and a soft segment, where the soft segment comprises two or more polycarbonate segments, polydimethylsiloxane segments, and polyalkyene oxide segments. In examples, a PU polymer is provided with a hard segment of about 35-45 weight percent, and a soft segment (remainder weight percent+up to 10 weight percent chain extender), where the soft segment comprises two or more polycarbonate segments, polydimethylsiloxane segments, and polyalkyene oxide segments. In examples, the soft segment comprises 35-45 weight percent polycarbonate segments and 15-20 weight percent polydimethylsiloxane segments, the remainder weight percent being hard segment and chain extender. In other examples, the soft segment comprises 35-45 weight percent polyakylene segments and 15-20 weight percent polydimethylsiloxane segments the remainder weight percent being hard segment and chain extender. In other examples, the soft segment comprises 35-45 weight percent total of both polyakylene segments and polycarbonate segments, and 15-20 weight percent polydimethylsiloxane segments the remainder weight percent being hard segment and chain extender. In examples, the polyalkylene segment comprises poly(tetramethylene oxide) (PTMO). In examples, PU polymer is provided with a hard segment and a soft segment, where the soft segment comprises two or more polycarbonate segments, polydimethylsiloxane segments, and polyalkyene oxide segments blended with a polyvinylpyrrolidone (PVP).

In examples, a diffusion resistance layer (RL) of the presently disclosed continuous levodopa monitor (“CLM”) includes the aforementioned PU polymer and/or PU polymer-PVP blend that provides stable, predicable levodopa and oxygen permeation and blocks at least some interfering agents. It will be appreciated that the hard/soft segment chemical composition, weight percentage of hard/soft segment, topology and block length distribution will impact the RL phase separation, hard segment/soft segment interaction, levodopa permeability, oxygen permeability, solubility of RL formulation for coating/dispensing and drying/curing processes and thus, influence sensor performance and stability.

In other examples, materials that forms the basis of the matrix of the resistance domain can be any of those known in the art as appropriate for use as membranes in sensor devices and as having sufficient permeability to allow relevant compounds to pass through it, for example, to allow an oxygen molecule to pass through the membrane from the sample under examination in order to reach the active enzyme or electrochemical electrodes. Examples of materials which can be used to make non-polyurethane type membranes include vinyl polymers, polyethers, polyesters, polyamides, inorganic polymers such as polysiloxanes and polycarbosiloxanes, natural polymers such as cellulosic and protein-based materials, poly(vinyl alcohol)-quaternized stilbazol (PVA-SbQ), and mixtures or combinations thereof.

In examples, the resistance domain is deposited onto the enzyme domain to yield a domain thickness of from about 0.5 micron or less to about 20 microns or more, more preferably from about 0.5, 0.1, 0.15, 0.2, 0.25, 0.3, 0.35, 0.4, 0.45, 0.5, 1, 1.5, 2, 2.5, 3, or 3.5 to about 4, 5, 6, 7, 8, 9, 10, 11, 12, 13, 14, 15, 16, 17, 18, 19, or 19.5 microns, and more preferably from about 2, 2.5 or 3 microns to about 3.5, 4, 4.5, or 5 microns. Preferably, the resistance domain is deposited onto the enzyme domain by spray coating or dip coating. In certain examples, spray coating is the preferred deposition technique. The spraying process atomizes and mists the solution, and therefore most or all of the solvent is evaporated prior to the coating material settling on the underlying domain, thereby minimizing contact of the solvent with the enzyme.

In examples, the resistance domain is deposited on the enzyme domain by spray-coating a solution of from about 1 wt. % to about 5 wt. % polymer and from about 95 wt. % to about 99 wt. % solvent. In spraying a solution of resistance domain material, including a solvent, onto the enzyme domain, it is desirable to mitigate or substantially reduce any contact with enzyme of any solvent in the spray solution that can deactivate the underlying enzyme of the enzyme domain. Tetrahydrofuran (THF) is one solvent that minimally or negligibly affects the enzyme of the enzyme domain upon spraying. Other solvents can also be suitable for use, as is appreciated by one skilled in the art.

Although a variety of spraying or deposition techniques can be used, spraying the resistance domain material and rotating the sensor at least one time by 180° can provide adequate coverage by the resistance domain. Spraying the resistance domain material and rotating the sensor at least two times by 120 degrees provides even greater coverage (one layer of 360° coverage), thereby ensuring resistivity to L-DOPA, such as is described in more detail above.

In examples, the resistance domain is spray-coated and subsequently cured for a time of from about 15 to about 90 minutes at a temperature of from about 40 to about 60° C. (and can be accomplished under vacuum (e.g., 20 to 30 mmHg)). A cure time of up to about 90 minutes or more can be advantageous to ensure complete drying of the resistance domain. While not wishing to be bound by theory, it is believed that complete drying of the resistance domain aids in stabilizing the sensitivity of the L-DOPA sensor signal. It reduces drifting of the signal sensitivity over time, and complete drying is believed to stabilize performance of the L-DOPA sensor signal in lower oxygen environments.

Advantageously, sensors with the membrane system described herein, including an electrode domain and/or interference domain, an enzyme domain, and a resistance domain, provide stable signal response to increasing L-DOPA levels of from about 0 to about 40 μM, and sustained function (at least 90% signal strength) even at low oxygen levels (for example, at about 0.6 mg/L O2). While not wishing to be bound by theory, it is believed that the resistance domain provides sufficient resistivity, or the enzyme domain provides sufficient enzyme, such that oxygen limitations are seen at a much lower concentration of oxygen as compared to prior art sensors.

In examples, a sensor signal with a current in the picoampere range or less is provided, which is described in more detail elsewhere herein. However, the ability to produce a signal with a current in the picoampere range can be dependent upon a combination of factors, including the electronic circuitry design (e.g., A/D converter, bit resolution, and the like), the membrane system (e.g., permeability of the analyte through the resistance domain, enzyme concentration, and/or electrolyte availability to the electrochemical reaction at the electrodes), and the exposed surface area of the working electrode. For example, the resistance domain can be designed to be more or less restrictive to the analyte depending upon to the design of the electronic circuitry, membrane system, and/or exposed electroactive surface area of the working electrode.

Accordingly, in examples, the membrane system is designed with a sensitivity of from about 1 pA/mg/dL to about 100 pA/mg/dL, preferably from about 5 pA/mg/dL to 25 pA/mg/dL, and more preferably from about 4 to about 7 pA/mg/dL. While not wishing to be bound by any particular theory, it is believed that membrane systems designed with a sensitivity in the preferred ranges permit measurement of the analyte signal in low analyte and/or low oxygen situations. Namely, conventional analyte sensors have shown reduced measurement accuracy in low analyte ranges due to lower availability of the analyte to the sensor and/or have shown increased signal noise in high analyte ranges due to insufficient oxygen necessary to react with the amount of analyte being measured. While not wishing to be bound by theory, it is believed that the membrane systems of the present disclosure, in combination with the electronic circuitry design and exposed electrochemical reactive surface area design, support measurement of the analyte in the picoampere range or less, which enables an improved level of resolution and accuracy in both low and high analyte ranges not seen in the prior art.

In some examples, gated amperometric detection is used to provide greater interference tolerance and to amplify the signal produced by L-DOPA detection. In some examples, an analyte sensor circuit may be recurrently turned off and turned back on. During a period in which the sensor is turned off, an analyte (e.g., L-DOPA) continues to interact with a sensor enzyme, which develops a signal that may be sensed. For, when a sensor circuit is off, L-DOPA continues to react with polyphenol oxidase enzyme to produce dopaquinone, which accumulates. When the sensor circuit is turned on, the accumulated dopaquinone creates a much stronger signal than occurs without accumulation. Importantly, some interference materials do not exhibit such an accumulation effect, so the signal-to-noise (or background or interference) ratio is improved. Thus, while the presence of interference materials may cause an error in a L-DOPA sensor estimate (because the interference material(s) impacts the raw signal observed from the sensor), the impact of interference material(s) may be reduced by gating the analyte sensor circuit to increase the signal-to-noise ratio between the L-DOPA signal and the interfering material. Gated amperometry is described in more detail in co-pending U.S. Patent Publication No. 2020-0205702.

### Interference-Free Membrane Systems

In general, it is believed that appropriate solvents and/or deposition methods can be chosen for one or more of the domains of the membrane system that form one or more transitional domains such that interferants do not substantially permeate there through. Thus, sensors can be built without distinct or deposited interference domains, which are non-responsive to interferants. While not wishing to be bound by theory, it is believed that a simplified multilayer membrane system, more robust multilayer manufacturing process, and reduced variability caused by the thickness and associated oxygen and L-DOPA sensitivity of the deposited micron-thin interference domain can be provided.

### Biointerface Membrane/Layer

In examples, the sensor includes a porous material disposed over some portion thereof, which modifies the host's tissue response to the sensor. In some examples, the porous material surrounding the sensor advantageously enhances and extends sensor performance and lifetime by slowing or reducing cellular migration to the sensor and associated degradation that would otherwise be caused by cellular invasion if the sensor were directly exposed to the in vivo environment. Alternatively, the porous material can provide stabilization of the sensor via tissue ingrowth into the porous material in the long term. Suitable porous materials include silicone, polytetrafluoroethylene, expanded polytetrafluoroethylene, polyethylene-co-tetrafluoroethylene, polyolefin, polyester, polycarbonate, biostable polytetrafluoroethylene, homopolymers, copolymers, terpolymers of polyurethanes, polypropylene (PP), polyvinylchloride (PVC), polyvinylidene fluoride (PVDF), polyvinyl alcohol (PVA), polybutylene terephthalate (PBT), polymethylmethacrylate (PMMA), polyether ether ketone (PEEK), polyamides, polyurethanes, cellulosic polymers, poly(ethylene oxide), poly(propylene oxide) and copolymers and blends thereof, polysulfones and block copolymers thereof including, for example, di-block, tri-block, alternating, random and graft copolymers, as well as metals, ceramics, cellulose, hydrogel polymers, poly(2-hydroxyethyl methacrylate, pHEMA), hydroxyethyl methacrylate, (HEMA), polyacrylonitrile-polyvinyl chloride (PAN-PVC), high density polyethylene, acrylic copolymers, nylon, polyvinyl difluoride, polyanhydrides, poly(l-lysine), poly(L-lactic acid), hydroxyethylmethacrylate, hydroxyapeptite, alumina, zirconia, carbon fiber, aluminum, calcium phosphate, titanium, titanium alloy, nitinol, stainless steel, and CoCr alloy, or the like, such as are described in U.S. Pat. Nos. 7,875,293 and 7,192,450.

In some examples, the porous material surrounding the sensor provides unique advantages in vivo (e.g., one to 14 days) that can be used to enhance and extend sensor performance and lifetime. However, such materials can also provide advantages in the long term too (e.g., greater than 14 days). Particularly, the in vivo portion of the sensor (the portion of the sensor that is implanted into the host's tissue) is encased (partially or fully) in a porous material. The porous material can be wrapped around the sensor (for example, by wrapping the porous material around the sensor or by inserting the sensor into a section of porous material sized to receive the sensor). Alternately, the porous material can be deposited on the sensor (for example, by electrospinning of a polymer directly thereon). In yet other alternative examples, the sensor is inserted into a selected section of porous biomaterial. Other methods for surrounding the in vivo portion of the sensor with a porous material can also be used as is appreciated by one skilled in the art.

The porous material surrounding the sensor advantageously slows or reduces cellular migration to the sensor and associated degradation that would otherwise be caused by cellular invasion if the sensor were directly exposed to the in vivo environment. Namely, the porous material provides a barrier that makes the migration of cells towards the sensor more tortuous and therefore slower. It is believed that this reduces or slows the sensitivity loss normally observed over time.

In examples wherein the porous material is a high oxygen solubility material, such as porous silicone, the high oxygen solubility porous material surrounds some of or the entire in vivo portion of the sensor. In some examples, a lower ratio of oxygen-to-L-DOPA can be sufficient to provide excess oxygen by using a high oxygen soluble domain (for example, a silicone- or fluorocarbon-based material) to enhance the supply/transport of oxygen to the enzyme membrane and/or electroactive surfaces. It is believed that some signal noise normally seen by a conventional sensor can be attributed to an oxygen deficit. Silicone has high oxygen permeability, thus promoting oxygen transport to the enzyme layer. By enhancing the oxygen supply through the use of a silicone composition, for example, L-DOPA concentration can be less of a limiting factor. In other words, if more oxygen is supplied to the enzyme and/or electroactive surfaces, then more L-DOPA can also be supplied to the enzyme without creating an oxygen rate-limiting excess. While not being bound by any particular theory, it is believed that silicone materials provide enhanced bio-stability when compared to other polymeric materials such as polyurethane.

In another example, the porous material further comprises a bioactive agent that releases upon insertion. In examples, the porous structure provides access for L-DOPA permeation while allowing drug release/elute. In examples, as the bioactive agent releases/elutes from the porous structure, L-DOPA transport may increase, for example, so as to offset any attenuation of L-DOPA transport from the aforementioned immune response factors.

When used herein, the terms “membrane” and “matrix” are meant to be interchangeable. In these examples, the aforementioned porous material is a biointerface membrane comprising a first domain that includes an architecture, including cavity size, configuration, and/or overall thickness, that modifies the host's tissue response, for example, by creating a fluid pocket, encouraging vascularized tissue ingrowth, disrupting downward tissue contracture, resisting fibrous tissue growth adjacent to the device, and/or discouraging barrier cell formation. The biointerface membrane in examples covers at least the sensing mechanism of the sensor and can be of any shape or size, including uniform, asymmetrically, or axi-symmetrically covering or surrounding a sensing mechanism or sensor.

A second domain of the biointerface membrane is optionally provided that is impermeable to cells and/or cell processes. A bioactive agent is optionally provided that is incorporated into the at least one of the first domain, the second domain, the sensing membrane, or other part of the implantable device, wherein the bioactive agent is configured to modify a host tissue response. In examples, the biointerface includes a bioactive agent, the bioactive agent being incorporated into at least one of the first and second domains of the biointerface membrane, or into the device and adapted to diffuse through the first and/or second domains, in order to modify the tissue response of the host to the membrane.

Due to the small dimension(s) of the sensor (sensing mechanism) of the present disclosure, some conventional methods of porous membrane formation and/or porous membrane adhesion are inappropriate for the formation of the biointerface membrane onto the sensor as described herein. Accordingly, the following examples exemplify systems and methods for forming and/or adhering a biointerface membrane onto a small structured sensor as defined herein. For example, the biointerface membrane or release membrane of the present disclosure can be formed onto the sensor using techniques such as electrospinning, molding, weaving, direct-writing, lyophilizing, wrapping, and the like.

In examples wherein the biointerface is directly-written onto the sensor, a dispenser dispenses a polymer solution using a nozzle with a valve, or the like, for example as described in U.S. Publication No. 2004/0253365 A1. In general, a variety of nozzles and/or dispensers can be used to dispense a polymeric material to form the woven or non-woven fibers of the biointerface membrane.

### Drug Release Membrane/Layer-Inflammatory Response Control

In general, the inflammatory response to biomaterial implants can be divided into two phases. The first phase consists of mobilization of mast cells and then infiltration of predominantly polymorphonuclear (PMN) cells. This phase is termed the acute inflammatory phase. Over the course of days to weeks, chronic cell types that comprise the second phase of inflammation replace the PMNs. Macrophage and lymphocyte cells predominate during this phase. While not wishing to be bound by any particular theory, it is believed that restricting vasodilation and/or blocking pro-inflammatory signaling, short-term stimulation of vascularization, or short-term inhibition of scar formation or barrier cell layer formation, provides protection from scar tissue formation and/or reduces acute inflammation, thereby providing a stable platform for sustained maintenance of the altered foreign body response, for example.

Accordingly, bioactive intervention can modify the foreign body response in the early weeks of foreign body capsule formation and alter the extended behavior of the foreign body capsule. Additionally, it is believed that in some circumstances the biointerface membranes of the present disclosure can benefit from bioactive intervention to overcome sensitivity of the membrane to implant procedure, motion of the implant, or other factors, which are known to otherwise cause inflammation, scar formation, and hinder device function in vivo.

In general, bioactive agents that are believed to modify tissue response include anti-inflammatory agents, anti-infective agents, anti-proliferative agents, anti-histamine agents, anesthetics, inflammatory agents, growth factors, angiogenic (growth) factors, adjuvants, immunosuppressive agents, antiplatelet agents, anticoagulants, ACE inhibitors, cytotoxic agents, anti-barrier cell compounds, vascularization compounds, anti-sense molecules, and the like. In some examples, preferred bioactive agents include S1P (Sphingosine-1-phosphate), Monobutyrin, Cyclosporin A, Anti-thrombospondin-2, Rapamycin (and its derivatives), NLRP3 inflammasome inhibitors such as MCC950, pilocarpine, and dexamethasone and/or dexamethasone acetate. However, other bioactive agents, biological materials (for example, proteins), or even non-bioactive substances can incorporated into the membranes of the present disclosure.

Bioactive agents suitable for use in the present disclosure are loosely organized into two groups: anti-barrier cell agents and vascularization agents. These designations reflect functions that are believed to provide short-term solute transport through the one or more membranes of the presently disclosed sensor, and additionally extend the life of a healthy vascular bed and hence solute transport through the one or more membranes long term in vivo. However, not all bioactive agents can be clearly categorized into one or other of the above groups; rather, bioactive agents generally comprise one or more varying mechanisms for modifying tissue response and can be generally categorized into one or both of the above-cited categories.

In examples, pilocarpine, dexamethasone, dexamethasone salts, or dexamethasone derivatives in particular, dexamethasone acetate, which, for example, abates the intensity of the FBC response at the device-tissue interface, is incorporated into any of the previously disclosed membranes, in the biointerfacing membrane or is provided in a separate membrane adjacent the biointerfacing membrane.

In another example, a combination of dexamethasone and dexamethasone acetate is incorporated into the drug releasing membrane. In another example, dexamethasone and/or dexamethasone acetate combined with one or more other anti-inflammatory and/or immunosuppressive agents is incorporated into the drug releasing membrane. Alternatively, Rapamycin, which is a potent specific inhibitor of some macrophage inflammatory functions, can be incorporated into the release membrane alone or in combination with dexamethasone, dexamethasone salts, dexamethasone derivatives in particular, dexamethasone acetate.

Other suitable medicaments, pharmaceutical compositions, therapeutic agents, or other desirable substances can be incorporated into the drug releasing membrane of the present disclosure, including, but not limited to, anti-inflammatory agents, anti-infective agents, necrosing agents, and anesthetics. It is to be understood that the different membrane/membrane systems described above can be applied to any of the sensors/sensor systems described herein. Additionally, it is also to be understood that any of the membranes (including membrane layers and domains), membrane properties, and membrane-derived results can be used with any of the sensors/sensor systems described herein.

Although the bioactive agent in some examples is incorporated into the biointerface membrane or release membrane and/or implantable device, in some examples the bioactive agent can be administered concurrently with, prior to, or after implantation of the device systemically, for example, by oral administration, or locally, for example, by subcutaneous injection near the implantation site. A combination of bioactive agent incorporated in the biointerface membrane and bioactive agent administration locally and/or systemically can be preferred in certain examples.

In examples, the drug release membrane functions as the biointerface membrane. In another example, the drug releasing membrane is chemically distinct from the biointerface membrane, or no biointerface membrane is used. In such examples, one or more bioactive agents are incorporated into the drug releasing membrane or both the biointerface membrane and the drug releasing membrane.

### Mediators

One or more mediators can be employed to facilitate the electrolysis of one or more analytes or of a second compound that correlates with or interferes with the signal transduction of the one or more analytes. Non-polymeric and polymeric redox mediators can be used in the presently disclosed continuous monitoring sensor.

In examples, zwitterionic compounds/polymers, Prussian blue, medola blue, methylene blue, methylene green, methyl viologen, ferrocyanide, ferrocene, cobalt ion and cobalt phthalocyanine can be used as a coating on one or more WEs to facilitate or otherwise assist in electron transfer and transduction of a detectable signal corresponding to one or more analytes. In examples, a transition metal complex is attached to one or more polymeric backbones as a redox mediator. In examples, the transition metal complexes include at least one substituted or unsubstituted biimidazole ligand. In another example, the transition metal complexes include at least one substituted or unsubstituted biimidazole ligand and a substituted or unsubstituted bipyridine or pyridylimidazole ligand. Suitable ligands for the metal complex mediators are inclusive of, for example, bipyridine, biimidazole, phenanthroline, or pyridyl(imidazole). Other bidentate ligands can be used for the metal complex mediators, for example, amino acids, oxalic acid, acetylacetone, diaminoalkanes, or o-diaminoarenes. Any combination of monodentate, bidentate, tridentate, tetradentate, or higher denticity ligands, for the metal complex mediators can be employed.

In examples the mediator is one or more metal compounds or metal complexes of ruthenium, osmium, iron (e.g., polyvinylferrocene or hexacyanoferrate), or cobalt, including metallocene compounds thereof, for example. In examples, the mediator is coupled or otherwise bound to the conductive material of any one of the reference or working or counter electrode. In examples, non-polymeric or polymeric mediator can be adsorbed on or covalently bound to the conductive material of the electrode, such as a carbon surface or surfaces of gold, platinum, palladium, rhodium and alloys thereof. In examples, the mediator is quaternized.

A variety of methods may be used to immobilize a polymeric or non-polymeric mediator on an electrode surface, for example, adsorptive immobilization with or without cross-linking, vapor depositing, functionalization of at least a portion of the electrode surface and then chemical bonding, (ionically or covalently), of the mediator polymer to the functional groups on the electrode surface. In examples, poly(4-vinylpyridine) or poly vinylpyridine-co-styrene or polyvinylimidazoles are at least in part complexed with a transition metal compound, such as [Os(bpy)2 Cl]^{+/2+} where bpy is 2,2′-bipyridine. In examples, at least a part of the pyridine rings of the poly(4-vinylpyridine) or poly vinylpyridine-co-styrene are reacted with 2-bromoethylamine, then crosslinked, for example, using a diepoxide, such as polyethylene glycol diglycidyl ether. Other polymeric and/or non-polymeric mediators can be used, such as PVI- and PVP-Ruthenium(phenanthroline dione).

Carbon surfaces can be modified for attachment of one or more polymeric and/or non-polymeric mediators, for example, by electroreduction of a diazonium salt, followed by activated by a carbodiimide, such as 1-ethyl-3-(3-dimethylaminopropyl)-carbodiimide hydrochloride then bound with a amine-functionalized mediator, such as the osmium-containing polymer described above, or 2-aminoethylferrocene, to form the mediator couple.

Similarly, gold can be functionalized by a thiol or an amine, such as cysteamine and mediator [Os(bpy)2 (pyridine-4-carboxylate)Cl]0/+can be activated by 1-ethyl-3-(3-dimethylaminopropyl)-carbodiimide hydrochloride to form a reactive O-acylisourea that reacts with the gold-bound amine to form an amide.

As employed herein, the following abbreviations apply: Eq and Eqs (equivalents); mEq (milliequivalents); M (molar); mM (millimolar) μM (micromolar); N (Normal); mol (moles); mmol (millimoles); μmol (micromoles); nmol (nanomoles); g (grams); mg (milligrams); μg (micrograms); Kg (kilograms); L (liters); mL (milliliters); dL (deciliters); μL (microliters); cm (centimeters); mm (millimeters); μm (micrometers); nm (nanometers); h and hr (hours); min. (minutes); s and sec. (seconds); ° C. (degrees Centigrade).

### Sequence Listings

polyphenol oxidase (PPO) enzyme (Agaricus bisporus) (Amino Acid
Residues 1-392):
SEQ ID NO: 1
MSDKKSLMPL VGIPGEIKNR LNILDFVKND KFFTLYVRAL QVLQARDQSD YSSFFQLGGI
HGLPYTEWAK AQPQLHLYKA NYCTHGTVLF PTWHRAYEST WEQTLWEAAG TVAQRFTTSD
QAEWIQAAKD LRQPFWDWGY WPNDPDFIGL PDQVIRDKQV EITDYNGTKI EVENPILHYK
FHPIEPTFEG DFAQWQTTMR YPDVQKQENI EGMIAGIKAA APGFREWTFN MLTKNYTWEL
FSNHGAVVGA HANSLEMVHN TVHFLIGRDP TLDPLVPGHM GSVPHAAFDP IFWMHHCNVD
RLLALWQTMN YDVYVSEGMN REATMGLIPG QVLTEDSPLE PFYTKNQDPW QSDDLEDWET
LGFSYPDFDP VKGKSKEEKS VYINDWVHKH YG.
(polyphenol oxidase (PPO) enzyme (Tyrosinase -Agaricus bisporus)
(Amino Acid Residues 1-576))
SEQ ID NO: 2
MSDKKSLMPLVGIPGEIKNRLNILDFVKNDKFFTLYVRALQVLQARDQSDYSSFFQLGGIHGLPY
TEWAKAQPQLHLYKANYCTHGTVLFPTWHRAYESTWEQTLWEAAGTVAQRFTTSDQAEWIQAAKDLRQP
FWDWGYWPNDPDFIGLPDQVIRDKQVEITDYNGTKIEVENPILHYKFHPIEPTFEGDFAQWQTTMRYPDVQ
KQENIEGMIAGIKAAAPGFREWTFNMLTKNYTWELFSNHGAVVGAHANSLEMVHNTVHFLIGRDPTLDPL
VPGHMGSVPHAAFDPIFWMHHCNVDRLLALWQTMNYDVYVSEGMNREATMGLIPGQVLTEDSPLEPFY
TKNQDPWQSDDLEDWETLGFSYPDFDPVKGKSKEEKSVYINDWVHKHYGFVTTQTENPALRLLSSFQRAKS
DHETQYALYDWVIHATFRYYELNNSFSIIFYFDEGEGCTLESIIGTVDAFRGTTSENCANCARSQDLIAEGFVHL
NYYIGCDIGQHADHEDDAVPLYEPTRVKEYLKKRKIGCKVVSAEGELTSLVVEIKGAPYYLPVGEARPKLDHEK
PIVILDDIIHRVN
-(polyphenol oxidase (PPO) enzyme (mutated Tyrosinase N260G -
Agaricus bisporus) (Amino Acid Residues 1-576))
SEQ ID NO: 3
MSDKKSLMPLVGIPGEIKNRLNILDFVKNDKFFTLYVRALQVLQARDQSDYSSFFQLGGIHGLPY
TEWAKAQPQLHLYKANYCTHGTVLFPTWHRAYESTWEQTLWEAAGTVAQRFTTSDQAEWIQAAKDLRQP
FWDWGYWPNDPDFIGLPDQVIRDKQVEITDYNGTKIEVENPILHYKFHPIEPTFEGDFAQWQTTMRYPDVQ
KQENIEGMIAGIKAAAPGFREWTFNMLTKNYTWELFSNHGAVVGAHANSLEMVHGTVHFLIGRDPTLDPL
VPGHMGSVPHAAFDPIFWMHHCNVDRLLALWQTMNYDVYVSEGMNREATMGLIPGQVLTEDSPLEPFY
TKNQDPWQSDDLEDWETLGFSYPDFDPVKGKSKEEKSVYINDWVHKHYGFVTTQTENPALRLLSSFQRAKS
DHETQYALYDWVIHATFRYYELNNSFSIIFYFDEGEGCTLESIIGTVDAFRGTTSENCANCARSQDLIAEGFVHL
NYYIGCDIGQHADHEDDAVPLYEPTRVKEYLKKRKIGCKVVSAEGELTSLVVEIKGAPYYLPVGEARPKLDHEK
PIVILDDIIHRVN
-(polyphenol oxidase (PPO) enzyme (mutated Tyrosinase P159Ins -
Agaricus bisporus) (Amino Acid Residues 1-577))
SEQ ID NO: 4
MSDKKSLMPLVGIPGEIKNRLNILDFVKNDKFFTLYVRALQVLQARDQSDYSSFFQLGGIHGLPY
TEWAKAQPQLHLYKANYCTHGTVLFPTWHRAYESTWEQTLWEAAGTVAQRFTTSDQAEWIQAAKDLRQP
FWDWGYWPNDPDFIGLPDQVIRDKQVEITDYNGTKIEVENPILHYKFHPIEPTFEGDFAQWQTTMRYPDVQ
KQENIEGMIAGIKAAAPGFREWTFNMLTKNYTWELFSNHGAVVGAHANSLEMVPHNTVHFLIGRDPTLDP
LVPGHMGSVPHAAFDPIFWMHHCNVDRLLALWQTMNYDVYVSEGMNREATMGLIPGQVLTEDSPLEPFY
TKNQDPWQSDDLEDWETLGFSYPDFDPVKGKSKEEKSVYINDWVHKHYGFVTTQTENPALRLLSSFQRAKS
DHETQYALYDWVIHATFRYYELNNSFSIIFYFDEGEGCTLESIIGTVDAFRGTTSENCANCARSQDLIAEGFVHL
NYYIGCDIGQHADHEDDAVPLYEPTRVKEYLKKRKIGCKVVSAEGELTSLVVEIKGAPYYLPVGEARPKLDHEK
PIVILDDIIHRVN
-(polyphenol oxidase (PPO) enzyme (mutated Tyrosinase F264R -
Agaricus bisporus) (Amino Acid Residues 1-576))
SEQ ID NO: 5
MSDKKSLMPLVGIPGEIKNRLNILDFVKNDKFFTLYVRALQVLQARDQSDYSSFFQLGGIHGLPY
TEWAKAQPQLHLYKANYCTHGTVLFPTWHRAYESTWEQTLWEAAGTVAQRFTTSDQAEWIQAAKDLRQP
FWDWGYWPNDPDFIGLPDQVIRDKQVEITDYNGTKIEVENPILHYKFHPIEPTFEGDFAQWQTTMRYPDVQ
KQENIEGMIAGIKAAAPGFREWTFNMLTKNYTWELFSNHGAVVGAHANSLEMVHNTVHRLIGRDPTLDPL
VPGHMGSVPHAAFDPIFWMHHCNVDRLLALWQTMNYDVYVSEGMNREATMGLIPGQVLTEDSPLEPFY
TKNQDPWQSDDLEDWETLGFSYPDFDPVKGKSKEEKSVYINDWVHKHYGFVTTQTENPALRLLSSFQRAKS
DHETQYALYDWVIHATFRYYELNNSFSIIFYFDEGEGCTLESIIGTVDAFRGTTSENCANCARSQDLIAEGFVHL
NYYIGCDIGQHADHEDDAVPLYEPTRVKEYLKKRKIGCKVVSAEGELTSLVVEIKGAPYYLPVGEARPKLDHEK
PIVILDDIIHRVN
(DOPA 4,5-dioxygenase DODA; Portulaca grandiflora (rose moss)
(Amino Acid Residues 1-270):
SEQ ID NO: 6
MGVGKEVSFKESFFLSHGNPAMLADESFIARNFLLGWKKNVFPVKPKSILVVSAHWETDVPCV
SAGQYPNVIYDFTEVPASMFQMKYPAPGCPKLAKRVQELLIAGGFKSAKLDEERGFDHSSWVPLSMMCPEA
DIPVCQLSVQPGLDATHHFNVGRALAPLKGEGVLFIGSGGAVHPSDDTPHWFDGVAPWAAEFDQWEDAL
LEGRYEDVNNYQTKAPEGWKLAHPIPEHFLPLHVAMGAGGEKSKAELIYRTWDHGTLGYASYKFTSI.
(3,4-dihydroxyphenyl-acetaldehyde synthase DHPAA; Bombyx mori
(silk moth) (Amino Acid Residues 1-500):
SEQ ID NO: 7
MDANQFREFGRAVIDMLASYAENIRDYDVLPSVEPGYLLRALPESAPEQPEDWKDIMKDFNQS
IMPGVTHWQSPQFHAFYPSGSSFASIIGNMLSDGLAVVGFSWMASPACTELEVVTMNWLGKLLDLPEEFLN
CSSGPGGGVIQGSASEATLVGLLVAKDKTVRRFMNNNPDLDENEIKAKLVAYTSDQCNSSVEKAGLLGSMK
MKLLKADADGCLRGETLKRAIEEDKSQGLIPCYVVANLGTTGTCAFDPLHELGPICSEEDIWLHVDAAYAGAA
FLCPEYRHLMKGIEHSQSFVTNAHKWLPVNFDCSAMWVKNGYDITRAFDVQRIYLDDVKTTIKIPDYRHWQ
MPLGRRFRALKLWTVMRIYGAEGLKTHIRQQIELAQYFAKLVRADERFVIGPEPTMALVCFRLKDGDTITRQL
LENITQKKKVFMVAGTHRDRYVIRFVICSRLTKKEDVDYSWSQIKKETDLIYSDKIHNKAQIPALEQFTSRELCE
KSK
(3,4-dihydroxyphenyl-acetaldehyde synthase DHPAA; Drosophila
melanogaster (fruit)) fly) (Amino Acid Residues 1-510):
SEQ ID NO: 8
MDAKEFREFGKAAIDYIADYLENIRDDDVLPNVEPGYLLDLLPTEMPEEPEAWKDVLGDISRVIK
PGLTHWQSPHMHAYYPTSTSYPSIVGEMLASGFGVIGFSWICSPACTELEVVVMDWLAKFLKLPAHFQHAS
DGPGGGVIQGSASEAVLVAVLAAREQAVANYRESHPELSESEVRGRLVAYSSDQSNSCIEKAGVLAAMPIRLL
PAGEDFVLRGDTLRGAIEEDVAAGRIPVICVATLGTTGTCAYDDIESLSAVCEEFKVWLHVDAAYAGGAFALE
ECSDLRKGLDRVDSLNFNLHKFMLVNFDCSAMWLRDANKVVDSFNVDRIYLKHKHEGQSQIPDFRHWQIPL
GRRFRALKVWITFRTLGAEGLRNHVRKHIELAKQFEQLVLKDSRFELVAPRALGLVCFRPKGDNEITTQLLQRL
MDRKKIYMVKAEHAGRQFLRFVVCGMDTKASDIDFAWQEIESQLTDLQAEQSLVARKSGNVGDLAQHFQI
HLSTENATHEKSQ.
(3,4-dihydroxyphenyl-acetaldehyde synthase DHPAA; Aedes aegypti
(mosquito)) (Amino Acid Residues 1-521):
SEQ ID NO: 9
MANMDIDEFKEFGKAAIDFVADYLVNIRDRDVLPSVEPGYLHDLLPNEIPEKGDDWKTIMEEFK
RFIVPGLTHWQSPHFHAFYPSQTSYSSIVGETLAAGLGVVGFSWICSPVCTELEVIMMNWIGQLLNLPRCFLN
CDEGNGGGVIQGSASESIFIAVLVAREQAVRRLKNEHPELTEAEIRGRLVAYTSDQSNSAVEKSGILGAIKMRL
LPADDDCVLRGRTLKKAVEEDKANGLFPVIMVATLGTTGTCAYDNLEEIGPYCNDNKLWLHVDAAYAGASF
CLPEYAWIKKGLEMADSLNFNLHKWLFVNFDCCAMWFKDAAMITEAFSVDRIYLQHKFQGMSKAPDYRH
WQIQLGRRFRSLKVWITLKTMGAEKIRELIRFHISLAQKFEQYVRADPRFEVTSSTLALVCFRLKGEDTYSKQLL
DNIVKRKKIYMIPATYQGKFILRFMIAGIDPQAEDIDYAWNEVKSQTDLLLGVDDNGNNVCSKKLIKEEIFEKD
NPVGKITESLGGLVLANEKAQ.

While certain examples of the present disclosure have been illustrated with reference to specific combinations of elements, various other combinations may also be provided without departing from the teachings of the present disclosure. Thus, the present disclosure should not be construed as being limited to the particular exemplary examples described herein and illustrated in the Figures, but may also encompass combinations of elements of the various illustrated examples and aspects thereof.

1.-89. (canceled)

90. A continuous monitoring sensor comprising:
an analyte sensing portion comprising a first working electrode, the first working electrode having an electrochemically active surface area, the analyte sensing portion configured to be at least partially implanted; and
at least one membrane adjacent the electrochemically active surface area, the at least one membrane comprising an enzyme domain, wherein the enzyme domain comprises at least one L-DOPA responsive enzyme configured to generate at least a first signal corresponding to an L-DOPA concentration.

91. The continuous monitoring sensor of claim 90, wherein the analyte sensing portion is present on a separate wire substrate or separate planar substrate.

92. The continuous monitoring sensor of claim 90, wherein the analyte sensing portion comprises a second working electrode electrically isolated from the first working electrode.

93. The continuous monitoring sensor of claim 90, wherein the analyte sensing portion comprises a second working electrode electrically isolated from the first working electrode configured for detecting oxygen or other in situ electroactive species.

94. The continuous monitoring sensor of claim 90, further comprising at least one membrane or one or more layers configured for buffering of the enzyme domain.

95. The continuous monitoring sensor of claim 90, further comprising at least one membrane or one or more layers configured for protonation of L-DOPAquinone.

96. The continuous monitoring sensor of claim 90, further comprising at least one membrane or one or more layers configured for attenuation of signal-interfering electroactive species to the electrochemically active surface area.

97. The continuous monitoring sensor of claim 90, further comprising at least one membrane or one or more layers, independently, are configured for protonation of L-DOPAquinone and oxygen diffusion to the electrochemically active surface area.

98. The continuous monitoring sensor of claim 90, wherein the at least one membrane or the one or more layers independently comprise a polymer chain having both hydrophilic and hydrophobic regions.

99. The continuous monitoring sensor of claim 97, wherein the at least one membrane or the one or more layers independently comprise a polymer chain having polyurethane and/or polyurea segments.

100. The continuous monitoring sensor of claim 99, wherein the polyurethane and/or polyurea segments comprise hard segments and soft segments.

101. The continuous monitoring sensor of claim 100, wherein the soft segments comprise poly(tetramethylene oxide) repeating units and/or polydialkylsiloxane repeating units.

102. The continuous monitoring sensor of claim 97, wherein the at least one membrane or the one or more layers independently comprise a polyurethane and/or polyurea polymer blended with polyvinylpyrrolidone.

103. The continuous monitoring sensor of claim 97, wherein the at least one membrane or the one or more layers independently comprise a polyelectrolyte polymer or a polymerized monomer comprising a zwitterionic functional group.

104. The continuous monitoring sensor of claim 103, wherein the polyelectrolyte polymer or the zwitterionic functional group is configured for protonation of L-DOPA-quinone present about the analyte sensing portion.

105. The continuous monitoring sensor of claim 90, further comprising a coating comprising a polymer with a heterocyclic group.

106. The continuous monitoring sensor of claim 97, wherein the at least one membrane or the one or more layers independently comprise a coating comprising a polymer chain having poly(1-vinyl imidazole), poly(4-vinyl pyridine), poly(2-vinyl pyridine), acrylonitrile, acrylamide, and/or copolymers or quaternized forms thereof.

107. The continuous monitoring sensor of claim 90, wherein the at least one L-DOPA responsive enzyme is a tyrosinase enzyme with specificity towards l-3,4-dihydroxyphenylalanine (L-DOPA).

108. The continuous monitoring sensor of claim 90, wherein the at least one L-DOPA responsive enzyme is a mutated tyrosinase enzyme with specificity towards l-3,4-dihydroxyphenylalanine (L-DOPA).

109. The continuous monitoring sensor of claim 90, wherein the at least one L-DOPA responsive enzyme is a synthase or a mutated synthase enzyme with specificity towards l-3,4-dihydroxyphenylalanine (L-DOPA).
